(19) United States
(12) Patent Application Publication
(10) Pub. No.: US 2022/0315558 A1
(43) Pub. Date: Oct. 6, 2022

(54) POLYMORPHS OF 1-(2-((((TRANS)-3-FLUORO-1-(3-FLUOROPYRIDIN-2-YL)CYCLOBUTYL)METHYL)AMINO)PYRIMIDIN-5-YL)-1H-PYRROLE-3-CARBOXAMIDE

(71) Applicant: Cytokinetics, Inc., South San Francisco, CA (US)

(72) Inventors: Bradley P. MORGAN, Oakland, CA (US); Matthew W. PETERSON, San Diego, CA (US)

(21) Appl. No.: 17/621,950

(22) PCT Filed: Jun. 26, 2020
(86) PCT No.: PCT/US2020/039906

§ 371 Date: Dec. 22, 2021

Related U.S. Application Data
(60) Provisional application No. 62/867,834, filed on Jun. 27, 2019.

(51) Int. Cl. C07D 401/14

(52) U.S. Cl. CPC C07D 401/14; C07B 2200/13

(57) ABSTRACT

Provided herein are polymorphs of 1-(2-4((trans)-3-fluoro-1-(3-fluoropy-ridin-2-yl)cyclobutyl)methyl)amino)pyrimidin-5-yl)-1H-pyrrole-3-carboxamide (shown below), compositions thereof, methods of preparation thereof, and methods of their uses.

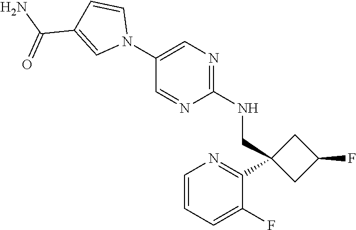 [SMILES: NC(=O)c1ccn(-c2cnc(NC[C@]3(c4ncccc4F)C[C@H](F)C3)nc2)c1]

### CROSS REFERENCE TO RELATED APPLICATION

This application claims the benefits of priority to U.S. Provisional Application No. 62/867,834, filed on Jun. 27, 2019, which is incorporated herein by reference in its entirety.

### FIELD

Provided herein are polymorphs of 1-(2-((((trans)-3-fluoro-1-(3-fluoropyridin-2-yl)cyclobutyl)methyl)amino)pyrimidin-5-yl)-1H-pyrrole-3-carboxamide, compositions thereof, methods of preparation thereof, and methods of their uses.

### BACKGROUND

The cytoskeleton of skeletal and cardiac muscle cells is unique compared to that of all other cells. It consists of a nearly crystalline array of closely packed cytoskeletal proteins called the sarcomere. The sarcomere is elegantly organized as an interdigitating array of thin and thick filaments. The thick filaments are composed of myosin, the motor protein responsible for transducing the chemical energy of ATP hydrolysis into force and directed movement. The thin filaments are composed of actin monomers arranged in a helical array. There are four regulatory proteins bound to the actin filaments, which allows the contraction to be modulated by calcium ions. An influx of intracellular calcium initiates muscle contraction; thick and thin filaments slide past each other driven by repetitive interactions of the myosin motor domains with the thin actin filaments.

Of the thirteen distinct classes of myosin in human cells, the myosin-II class is responsible for contraction of skeletal, cardiac, and smooth muscle. This class of myosin is significantly different in amino acid composition and in overall structure from myosin in the other twelve distinct classes. Myosin-II forms homo-dimers resulting in two globular head domains linked together by a long alpha-helical coiled-coiled tail to form the core of the sarcomere's thick filament. The globular heads have a catalytic domain where the actin binding and ATPase functions of myosin take place. Once bound to an actin filament, the release of phosphate (cf. ADP-Pi to ADP) signals a change in structural conformation of the catalytic domain that in turn alters the orientation of the light-chain binding lever arm domain that extends from the globular head; this movement is termed the powerstroke. This change in orientation of the myosin head in relationship to actin causes the thick filament of which it is a part to move with respect to the thin actin filament to which it is bound. Un-binding of the globular head from the actin filament (Ca²⁺ regulated) coupled with return of the catalytic domain and light chain to their starting conformation/orientation completes the catalytic cycle, responsible for intracellular movement and muscle contraction.

Tropomyosin and troponin mediate the calcium effect on the interaction on actin and myosin. The troponin complex is comprised of three polypeptide chains: troponin C, which binds calcium ions; troponin I, which binds to actin; and troponin T, which binds to tropomyosin. The skeletal troponin-tropomyosin complex regulates the myosin binding sites extending over several actin units at once.

Troponin, a complex of the three polypeptides described above, is an accessory protein that is closely associated with actin filaments in vertebrate muscle. The troponin complex acts in conjunction with the muscle form of tropomyosin to mediate the Ca²⁺ dependency of myosin ATPase activity and thereby regulate muscle contraction. The troponin polypeptides T, I, and C, are named for their tropomyosin binding, inhibitory, and calcium binding activities, respectively. Troponin T binds to tropomyosin and is believed to be responsible for positioning the troponin complex on the muscle thin filament. Troponin I binds to actin, and the complex formed by troponins I and T, and tropomyosin inhibits the interaction of actin and myosin. Skeletal troponin C is capable of binding up to four calcium molecules. Studies suggest that when the level of calcium in the muscle is raised, troponin C exposes a binding site for troponin I, recruiting it away from actin. This causes the tropomyosin molecule to shift its position as well, thereby exposing the myosin binding sites on actin and stimulating myosin ATPase activity.

U.S. Pat. No. 8,962,632, which is herein incorporated by reference in its entirety, discloses 1-(2-((((trans)-3-fluoro-1-(3-fluoropyridin-2-yl)cyclobutyl)methyl)amino)pyrimidin-5-yl)-1H-pyrrole-3-carboxamide, a next-generation fast skeletal muscle troponin activator (FSTA) as a potential treatment for people living with debilitating diseases and conditions associated with neuromuscular or non-neuromuscular dysfunction, muscular weakness, and/or muscle fatigue.

To move a drug candidate such as 1-(2-((((trans)-3-fluoro-1-(3-fluoropyridin-2-yl)cyclobutyl)methyl)amino)pyrimidin-5-yl)-1H-pyrrole-3-carboxamide to a viable pharmaceutical product, it can be important to understand whether the drug candidate has polymorph forms, as well as the relative stability and interconversions of these forms under conditions likely to be encountered upon large-scale production, transportation, storage and pre-usage preparation. The ability to control and produce a stable polymorph with a robust manufacturing process can be key for regulatory approval and marketing. Large scale production processes for high purity 1-(2-((((trans)-3-fluoro-1-(3-fluoropyridin-2-yl)cyclobutyl)methyl)amino)pyrimidin-5-yl)-1H-pyrrole-3-carboxamide can be improved by use of particular polymorphic forms. Accordingly, there is a need for various new crystalline forms of 1-(2-((((trans)-3-fluoro-1-(3-fluoropyridin-2-yl)cyclobutyl)methyl)amino)pyrimidin-5-yl)-1H-pyrrole-3-carboxamide with different chemical and physical stabilities, and formulations and uses of the same.

### BRIEF SUMMARY

In one aspect, provided herein are polymorphs of 1-(2-((((trans)-3-fluoro-1-(3-fluoropyridin-2-yl)cyclobutyl)methyl)amino)pyrimidin-5-yl)-1H-pyrrole-3-carboxamide.

In another aspect, provided herein are methods of preparing polymorphs of 1-(2-((((trans)-3-fluoro-1-(3-fluoropyridin-2-yl)cyclobutyl)methyl)amino)pyrimidin-5-yl)-1H-pyrrole-3-carboxamide.

In another aspect, provided herein are compositions containing the polymorphs of 1-(2-((((trans)-3-fluoro-1-(3-fluoropyridin-2-yl)cyclobutyl)methyl)amino)pyrimidin-5-yl)-1H-pyrrole-3-carboxamide as described herein.

In another aspect, provided herein are methods of treating a disease or condition associated with neuromuscular or non-neuromuscular dysfunction, muscular weakness, and/or muscle fatigue.

### DESCRIPTION OF THE DRAWINGS

FIG. 1A shows experimental and simulated X-ray powder diffraction (XRPD) patterns of polymorphic Form I of 1-(2-((((trans)-3-fluoro-1-(3-fluoropyridin-2-yl)cyclobutyl)methyl)amino)pyrimidin-5-yl)-1H-pyrrole-3-carboxamide.

FIG. 1B shows differential scanning calorimetry (DSC) and thermographic analysis (TGA) graphs of polymorphic Form I of 1-(2-((((trans)-3-fluoro-1-(3-fluoropyridin-2-yl)cyclobutyl)methyl)amino)pyrimidin-5-yl)-1H-pyrrole-3-carboxamide.

FIG. 1C shows a Gravimetric Vapour Sorption (GVS) graph of polymorphic Form I of 1-(2-((((trans)-3-fluoro-1-(3-fluoropyridin-2-yl)cyclobutyl)methyl)amino)pyrimidin-5-yl)-1H-pyrrole-3-carboxamide.

FIG. 1D shows XRPD patterns of polymorphic Form I of 1-(2-((((trans)-3-fluoro-1-(3-fluoropyridin-2-yl)cyclobutyl)methyl)amino)pyrimidin-5-yl)-1H-pyrrole-3-carboxamide after storage for 7 days at 40° C./75% RH and 25° C./97% RH.

FIG. 2A shows experimental and simulated XRPD patterns of polymorphic Form II of 1-(2-((((trans)-3-fluoro-1-(3-fluoropyridin-2-yl)cyclobutyl)methyl)amino)pyrimidin-5-yl)-1H-pyrrole-3-carboxamide.

FIG. 2B shows DSC and TGA graphs of polymorphic Form II of 1-(2-((((trans)-3-fluoro-1-(3-fluoropyridin-2-yl)cyclobutyl)methyl)amino)pyrimidin-5-yl)-1H-pyrrole-3-carboxamide.

FIG. 2C shows a GVS graph of polymorphic Form II of 1-(2-((((trans)-3-fluoro-1-(3-fluoropyridin-2-yl)cyclobutyl)methyl)amino)pyrimidin-5-yl)-1H-pyrrole-3-carboxamide.

FIG. 3A shows experimental and simulated XRPD patterns of polymorphic Form III of 1-(2-((((trans)-3-fluoro-1-(3-fluoropyridin-2-yl)cyclobutyl)methyl)amino)pyrimidin-5-yl)-1H-pyrrole-3-carboxamide dioxane solvate.

FIG. 3B shows DSC and TGA graphs of polymorphic Form III of 1-(2-((((trans)-3-fluoro-1-(3-fluoropyridin-2-yl)cyclobutyl)methyl)amino)pyrimidin-5-yl)-1H-pyrrole-3-carboxamide dioxane solvate.

FIG. 4 shows XRPD patterns of polymorphic Form IV of 1-(2-((((trans)-3-fluoro-1-(3-fluoropyridin-2-yl)cyclobutyl)methyl)amino)pyrimidin-5-yl)-1H-pyrrole-3-carboxamide prepared using different methods.

FIG. 5A shows an experimental XRPD pattern of polymorphic Form V of 1-(2-((((trans)-3-fluoro-1-(3-fluoropyridin-2-yl)cyclobutyl)methyl)amino)pyrimidin-5-yl)-1H-pyrrole-3-carboxamide.

FIG. 5B shows DSC and TGA graphs of polymorphic Form V of 1-(2-((((trans)-3-fluoro-1-(3-fluoropyridin-2-yl)cyclobutyl)methyl)amino)pyrimidin-5-yl)-1H-pyrrole-3-carboxamide.

FIG. 5C shows a GVS graph of polymorphic Form V of 1-(2-((((trans)-3-fluoro-1-(3-fluoropyridin-2-yl)cyclobutyl)methyl)amino)pyrimidin-5-yl)-1H-pyrrole-3-carboxamide.

FIG. 6 shows an XRPD pattern of Form II after heating at 150° C.

FIG. 7 shows the results of competitive slurry experiments between Form I and Form II.

FIG. 8 shows the results of competitive slurry experiments between Form I and Form IV.

FIG. 9 shows the results of competitive slurry experiments between Form I and Form V using ethanol in the temperature range from 4° C. to 60° C.

FIG. 10 shows the results of competitive slurry experiments between Form I and Form V using methanol in the temperature range from 4° C. to 60° C.

FIG. 11 shows the results of competitive slurry experiments between Form I and Form V in the temperature range from 65° C. to 75° C.

FIG. 12 shows an overlay of the XRPD patterns of Forms I-V (from top to bottom: Form V, Form IV, Form III, Form II, Form I).

### DETAILED DESCRIPTION

### Definitions

As used herein and in the appended claims, the singular forms “a”, “an” and “the” include plural forms, unless the context clearly dictates otherwise.

As used herein, and unless otherwise specified, the terms “about” and “approximately,” when used in connection with doses, amounts, or weight percent of ingredients of a composition or a dosage form, mean a dose, amount, or weight percent that is recognized by those of ordinary skill in the art to provide a pharmacological effect equivalent to that obtained from the specified dose, amount, or weight percent. Specifically, the terms “about” and “approximately,” when used in this context, contemplate a dose, amount, or weight percent within 15%, within 10%, within 5%, within 4%, within 3%, within 2%, within 1%, or within 0.5% of the specified dose, amount, or weight percent.

As used herein, the term “polymorph” or “polymorphic form” refers to a crystalline form of a compound. Different polymorphs may have different physical properties such as, for example, melting temperatures, heats of fusion, solubilities, dissolution rates, and/or vibrational spectra as a result of the arrangement or conformation of the molecules or ions in the crystal lattice. The differences in physical properties exhibited by polymorphs may affect pharmaceutical parameters, such as storage stability, compressibility, density (important in formulation and product manufacturing), and dissolution rate (an important factor in bioavailability). Differences in stability can result from changes in chemical reactivity (e.g., differential oxidation, such that a dosage form discolors more rapidly when comprised of one polymorph than when comprised of another polymorph), mechanical changes (e.g., tablets crumble on storage as a kinetically favored polymorph converts to thermodynamically more stable polymorph), or both (e.g., tablets of one polymorph are more susceptible to breakdown at high humidity). As a result of solubility/dissolution differences, in the extreme case, some polymorphic transitions may result in lack of potency or, at the other extreme, toxicity. In addition, the physical properties of a crystalline form may be important in processing; for example, one polymorph might be more likely to form solvates or might be difficult to filter and wash free of impurities (e.g., particle shape and size distribution might be different between polymorphs).

As used herein, “therapeutically effective amount” indicates an amount that results in a desired pharmacological and/or physiological effect for the condition. The effect may be prophylactic in terms of completely or partially preventing a condition or symptom thereof and/or may be therapeutic in terms of a partial or complete cure for the condition and/or adverse effect attributable to the condition.

As used herein, the term “pharmaceutically acceptable carrier,” and cognates thereof, refers to adjuvants, binders, diluents, etc. known to the skilled artisan that are suitable for administration to an individual (e.g., a mammal or non-mammal). Combinations of two or more carriers are also contemplated. The pharmaceutically acceptable carrier(s) and any additional components, as described herein, should be compatible for use in the intended route of administration (e.g., oral, parenteral) for a particular dosage form, as would be recognized by the skilled artisan.

The terms “treat,” “treating,” and “treatment” are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. Often, the beneficial effects that a subject derives from a therapeutic agent do not result in a complete cure of the disease, disorder or condition.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

As used herein, the term “substantially as shown in” when referring, for example, to an XRPD pattern, a DSC graph, a TGA graph, or a GVS graph, includes a pattern or graph that is not necessarily identical to those depicted herein, but that falls within the limits of experimental error or deviations when considered by one of ordinary skill in the art.

As used herein, the term “substantially free of” means that the composition comprising the polymorphic form contains less than 50%, less than 40%, less than 30%, less than 20%, less than 15%, less than 10%, less than 5%, less than 4%, less than 3%, less than 2%, or less than 1% by weight of the indicated substance or substances.

### Polymorphs

In one aspect, provided herein are polymorphs of 1-(2-((((trans)-3-fluoro-1-(3-fluoropyridin-2-yl)cyclobutyl)methyl)amino)pyrimidin-5-yl)-1H-pyrrole-3-carboxamide, a compound having the structure shown below,

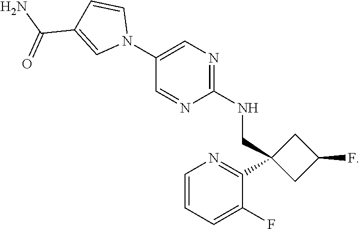 [SMILES: NC(=O)c1ccn(-c2cnc(NC[C@]3(c4ncccc4F)C[C@H](F)C3)nc2)c1]

In some embodiments, a polymorphic form provided may be a hydrate. In some embodiments, a polymorphic form provided may be a solvate. In some embodiments, the polymorphic form is a dioxane solvate. In some embodiments, the polymorphic form is a THF solvate. The polymorphs may have properties such as bioavailability and stability under certain conditions that are suitable for medical or pharmaceutical uses.

A polymorph of 1-(2-((((trans)-3-fluoro-1-(3-fluoropyridin-2-yl)cyclobutyl)methyl)amino)pyrimidin-5-yl)-1H-pyrrole-3-carboxamide may provide the advantages of bioavailability and stability and may be suitable for use as an active agent in a pharmaceutical composition. Variations in the crystal structure of a pharmaceutical drug substance may affect the dissolution rate (which may affect bioavailability, etc.), manufacturability (e.g., ease of handling, ease of purification, ability to consistently prepare doses of known strength, etc.) and stability (e.g., thermal stability, shelf life (including resistance to degradation), etc.) of a pharmaceutical drug product. Such variations may affect the methods of preparation or formulation of pharmaceutical compositions in different dosage or delivery forms, such as solid oral dosage forms including tablets and capsules. Compared to other forms such as non-crystalline or amorphous forms, polymorphs may provide desired or suitable hygroscopicity, particle size control, dissolution rate, solubility, purity, physical and chemical stability, manufacturability, yield, reproducibility, and/or process control. Thus, polymorphs of 1-(2-((((trans)-3-fluoro-1-(3-fluoropyridin-2-yl)cyclobutyl)methyl)amino)pyrimidin-5-yl)-1H-pyrrole-3-carboxamide may provide advantages of improving the manufacturing process of an active agent or the stability or storability of a drug product form of the active agent, or having suitable bioavailability and/or stability as an active agent.

The use of certain conditions, such as the use of different solvents and/or temperatures, has been found to produce different polymorphs of 1-(2-((((trans)-3-fluoro-1-(3-fluoropyridin-2-yl)cyclobutyl)methyl)amino)pyrimidin-5-yl)-1H-pyrrole-3-carboxamide, including polymorphic Forms I-V described herein, which may exhibit one or more favorable characteristics described herein. The processes for the preparation of the polymorphs described herein and characterization of these polymorphs are described in greater detail below.

### Form I

In some embodiments, provided herein is polymorphic Form I of 1-(2-((((trans)-3-fluoro-1-(3-fluoropyridin-2-yl)cyclobutyl)methyl)amino)pyrimidin-5-yl)-1H-pyrrole-3-carboxamide. Crystal structure information of Form I is provided in Table 1A.

TABLE 1A

| Molecular | C₁₉H₁₈F₂N₆O₁ | | | | |
|---|---|---|---|---|---|
| formula | | | | | |
| Molecular weight | 384.39 | | | | |
| Crystal system | Monoclinic | | | | |
| Space group | P2(1)/c | a | 9.8197(2) Å, | α | 90°, |
| | | b | 9.09520(10) Å, | β | 91.0970(10)°, |
| | | c | 20.0054(3) Å, | γ | 90° |

| | |
|---|---|
| V | 1786.40(5) Å³ |
| Z | 4 |
| D_{c} | 1.429 g · cm⁻¹ |
| μ | 0.909 mm⁻¹ |
| Source, λ | Mo—K(alpha), 1.54178 Å |
| F(000) | 800 |
| T | 100(1)K |
| Crystal | Colourless rhombus, 0.2 × 0.07 × 0.02 mm |
| Data truncated to | 0.80 Å |
| θₘₐₓ | 76.14° |
| Completeness | 99.60% |
| Reflections | 17454 |
| Unique reflections | 3725 |
| Rᵢₙₜ | 0.0305 |

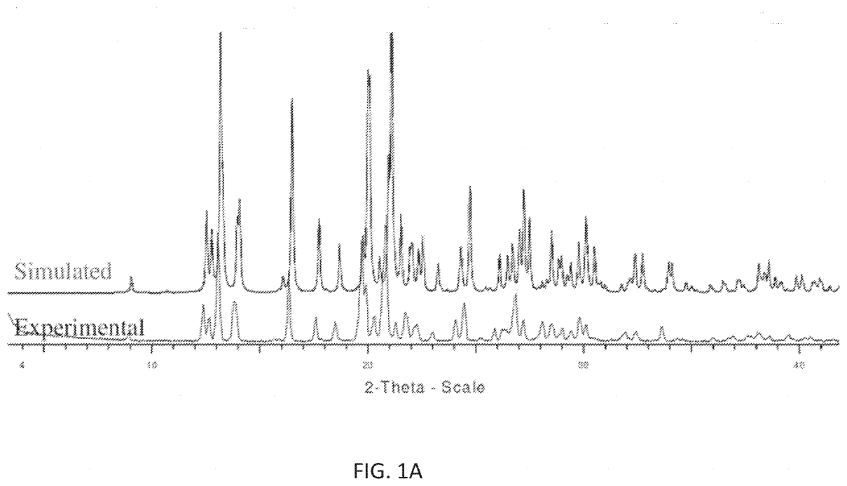

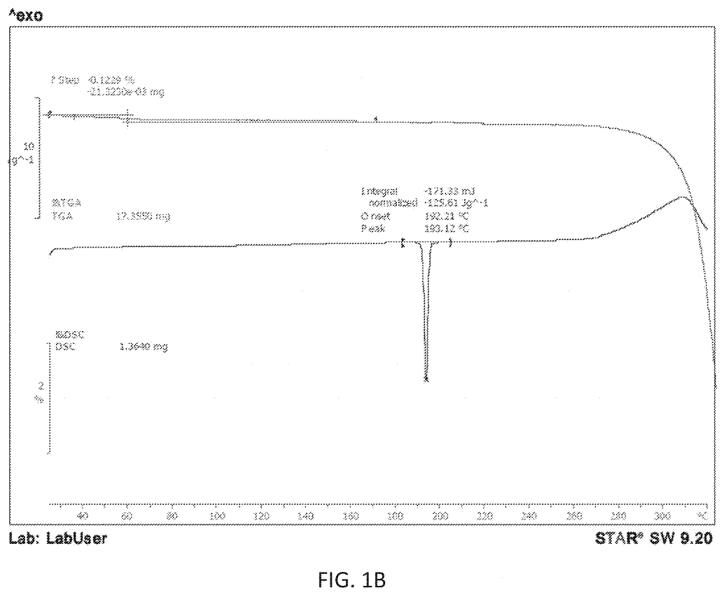

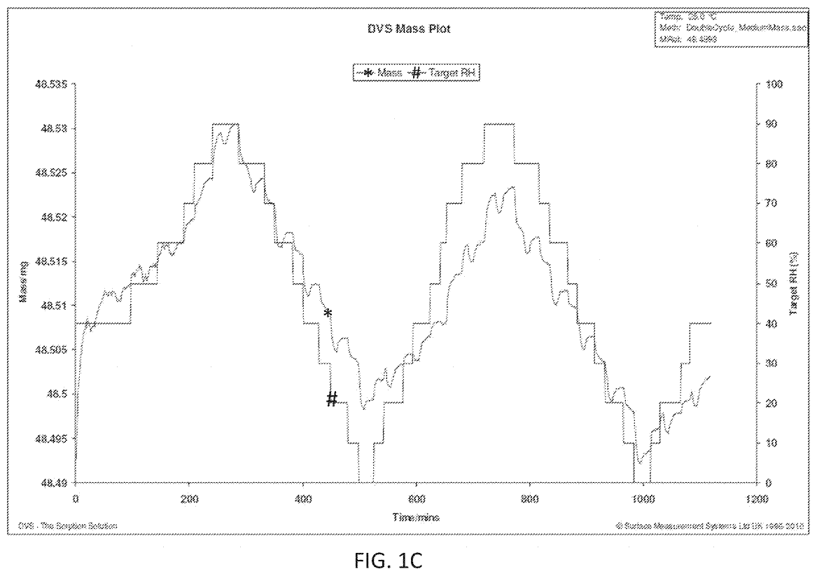

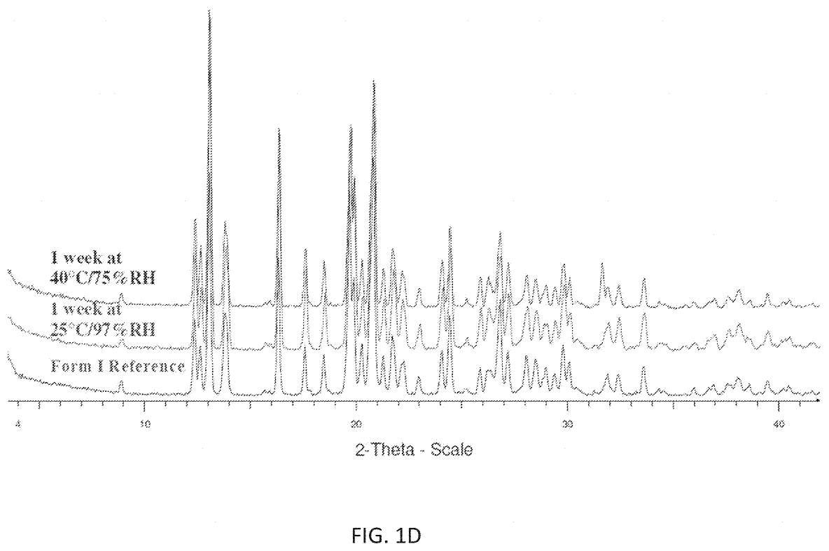

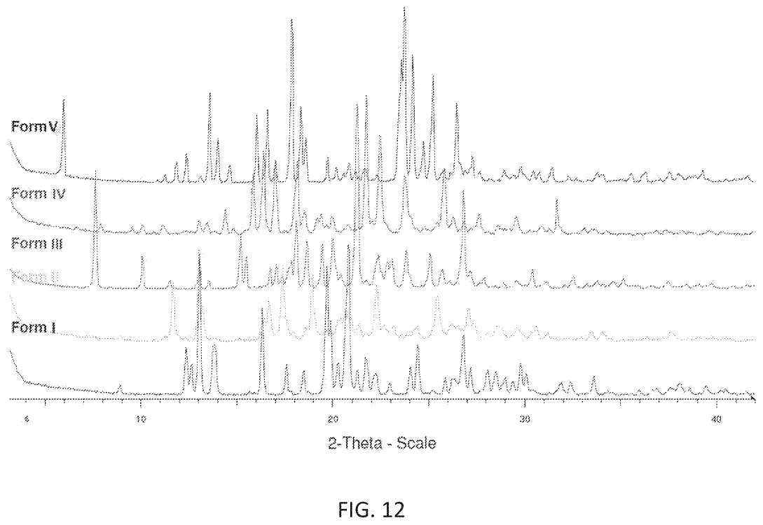

In some embodiments, Form I has an XRPD pattern substantially as shown in FIG. 1A or FIG. 12. In some embodiments, Form I has an XRPD pattern substantially as shown in FIG. 1A. In some embodiments, Form I has an XRPD pattern substantially as shown in FIG. 12.

Angles 2-theta and relative peak intensities that may be observed for Form I using XRPD are shown in Table 1B.

TABLE 1B

| Angle/2θ | d value/Å | Intensity/% |
|---|---|---|
| 8.9 | 9.9 | 8.3 |
| 12.3 | 7.2 | 33.1 |
| 12.6 | 7.0 | 22.3 |
| 13.0 | 6.8 | 93.4 |
| 13.8 | 6.4 | 35.5 |
| 16.3 | 5.4 | 58.6 |
| 17.6 | 5.0 | 22.2 |
| 18.4 | 4.8 | 18.4 |
| 19.7 | 4.5 | 86.2 |
| 19.9 | 4.5 | 50.0 |
| 20.3 | 4.4 | 23.1 |
| 20.8 | 4.3 | 100.0 |
| 21.3 | 4.2 | 18.0 |
| 21.7 | 4.1 | 26.5 |
| 22.2 | 4.0 | 16.1 |
| 23.0 | 3.9 | 9.6 |
| 24.1 | 3.7 | 20.3 |
| 24.5 | 3.6 | 34.6 |
| 25.9 | 3.4 | 13.2 |
| 26.3 | 3.4 | 12.4 |
| 26.8 | 3.3 | 41.4 |
| 27.2 | 3.3 | 20.1 |
| 28.1 | 3.2 | 18.2 |
| 28.5 | 3.1 | 17.2 |
| 29.0 | 3.1 | 13.3 |
| 29.4 | 3.0 | 10.9 |
| 29.8 | 3.0 | 22.4 |
| 30.1 | 3.0 | 15.5 |
| 31.9 | 2.8 | 10.6 |
| 32.4 | 2.8 | 10.5 |
| 33.7 | 2.7 | 14.5 |

In some embodiments, polymorphic Form I has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 1A or 12 or as provided in Table 1B. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form I, can vary by about ±0.6 degrees, ±0.4 degrees, ±0.2 degrees, or ±0.1 degrees 2-theta.

In some embodiments, polymorphic Form I has an XRPD pattern comprising peaks at angles 2-theta of 8.9±0.2, 12.3±0.2, 12.6±0.2, 13.0±0.2, 13.8±0.2, 16.3±0.2, 17.6±0.2, 18.4±0.2, 19.7±0.2, 19.9±0.2, 20.3±0.2, 20.8±0.2, 21.3±0.2, 21.7±0.2, 22.2±0.2, 23.0±0.2, 24.1±0.2, 24.5±0.2, 25.9±0.2, 26.3±0.2, 26.8±0.2, 27.2±0.2, 28.1±0.2, 28.5±0.2, 29.0±0.2, 29.4±0.2, 29.8±0.2, 30.1±0.2, 31.9±0.2, 32.4±0.2, and 33.7±0.2 degrees. In some embodiments, polymorphic Form I has an XRPD pattern comprising peaks at angles 2-theta of 12.3±0.2, 13.0±0.2, 13.8±0.2, 16.3±0.2, 19.7±0.2, 19.9±0.2, 20.8±0.2, 21.7±0.2, 24.5±0.2, and 26.8±0.2 degrees. In some embodiments, polymorphic Form I has an XRPD pattern comprising peaks at angles 2-theta of 13.0±0.2, 16.3±0.2, 19.7±0.2, 19.9±0.2, and 20.8±0.2 degrees. It is to be understood that additional peaks in the XRPD pattern other than those shown in FIG. 1A or 12 or as provided in Table 1B may be observed, for instance, due to the presence of impurities, solvent, or other polymorphs or amorphic forms present in the test sample.

In some embodiments, Form I has a differential scanning calorimetry (DSC) graph substantially as shown in FIG. 1B. In some embodiments, Form I is characterized as having a melting endotherm onset at about 192° C. as determined by DSC. In some embodiments, Form I is characterized as having a melting endotherm onset at 192±2° C. (e.g., 192±1.9° C., 192±1.8° C., 192±1.7° C., 192±1.6° C., 192±1.5° C., 192±1.4° C., 192±1.3° C., 192±1.2° C., 192±1, 192±0.9° C., 192±0.8° C., 192±0.7° C., 192±0.6° C., 192±0.5° C., 192±0.4° C., 192±0.3° C., 192±0.2° C., or 192±0.1° C.) as determined by DSC.

In some embodiments, Form I has a thermographic analysis (TGA) graph substantially as shown in FIG. 1B.

In some embodiments, Form I has a Gravimetric Vapour Sorption (GVS) graph substantially as shown in FIG. 1C.

In some embodiments, when stored for a period of 1 week under two different temperature/RH conditions (40° C./75% RH and 25° C./97% RH), Form I shows no changes as determined by XRPD.

In some embodiments of Form I, at least one, at least two, at least three, at least four, at least five, or all of the following (a)-(f) apply:

(a) Form I has an XRPD pattern comprising peaks at angles 2-theta of 13.0±0.2, 16.3±0.2, 19.7±0.2, 19.9±0.2, and 20.8±0.2 degrees; an XRPD pattern comprising peaks at angles 2-theta of 12.3±0.2, 13.0±0.2, 13.8±0.2, 16.3±0.2, 19.7±0.2, 19.9±0.2, 20.8±0.2, 21.7±0.2, 24.5±0.2, and 26.8±0.2 degrees; or an XRPD pattern comprising peaks at angles 2-theta of 8.9±0.2, 12.3±0.2, 12.6±0.2, 13.0±0.2, 13.8±0.2, 16.3±0.2, 17.6±0.2, 18.4±0.2, 19.7±0.2, 19.9±0.2, 20.3±0.2, 20.8±0.2, 21.3±0.2, 21.7±0.2, 22.2±0.2, 23.0±0.2, 24.1±0.2, 24.5±0.2, 25.9±0.2, 26.3±0.2, 26.8±0.2, 27.2±0.2, 28.1±0.2, 28.5±0.2, 29.0±0.2, 29.4±0.2, 29.8±0.2, 30.1±0.2, 31.9±0.2, 32.4±0.2, and 33.7±0.2 degrees;
(b) Form I has an XRPD pattern substantially as shown in FIG. 1A or FIG. 12;
(c) Form I has a DSC graph substantially as shown in FIG. 1B;
(d) Form I is characterized as having a melting endotherm onset at about 192° C. as determined by DSC;
(e) Form I has a TGA graph substantially as shown in FIG. 1B; and
(f) Form I has a GVS graph substantially as shown in FIG. 1C.

### Form II

In some embodiments, provided herein is polymorphic Form II of 1-(2-((((trans)-3-fluoro-1-(3-fluoropyridin-2-yl)cyclobutyl)methyl)amino)pyrimidin-5-yl)-1H-pyrrole-3-carboxamide. Crystal structure information of Form II is provided in Table 2A.

TABLE 2A

| Molecular | C₁₉H₁₈F₂N₆O₁ | | | | |
|---|---|---|---|---|---|
| formula | | | | | |
| Molecular weight | 384.39 | | | | |
| Crystal system | Monoclinic | | | | |
| Space group | P2(1)/n | a | 13.0790(6) Å, | α | 90°, |
| | | b | 9.0994(4) Å, | β | 92.620(5)°, |
| | | c | 15.1004(7) Å, | γ | 90° |

| | |
|---|---|
| V | 1795.24(14) Å³ |
| Z | 4 |
| D_{c} | 1.422 g · cm⁻¹ |
| μ | 0.904 mm⁻¹ |
| Source, λ | Cu—K(alpha), 1.54178 Å |
| F(000) | 800 |
| T | 100(1)K |
| Crystal | Colourless plate, 0.2 × 0.2 × 0.07 mm |
| Data truncated to | 0.80 Å |
| θₘₐₓ | 74.48° |
| Completeness | 98.7% |
| Reflections | 8270 |
| Unique reflections | 3618 |
| Rᵢₙₜ | 0.0279 |

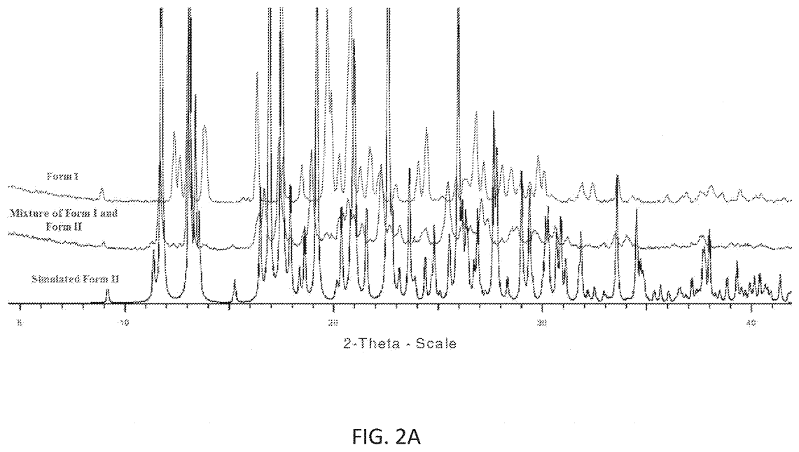

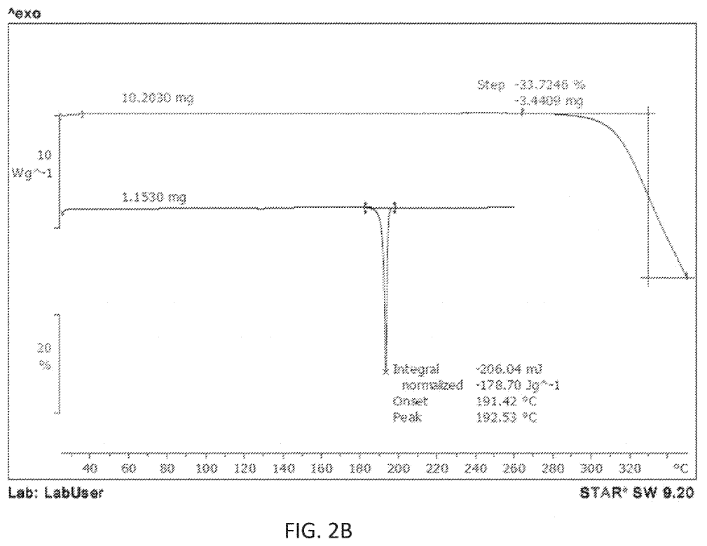

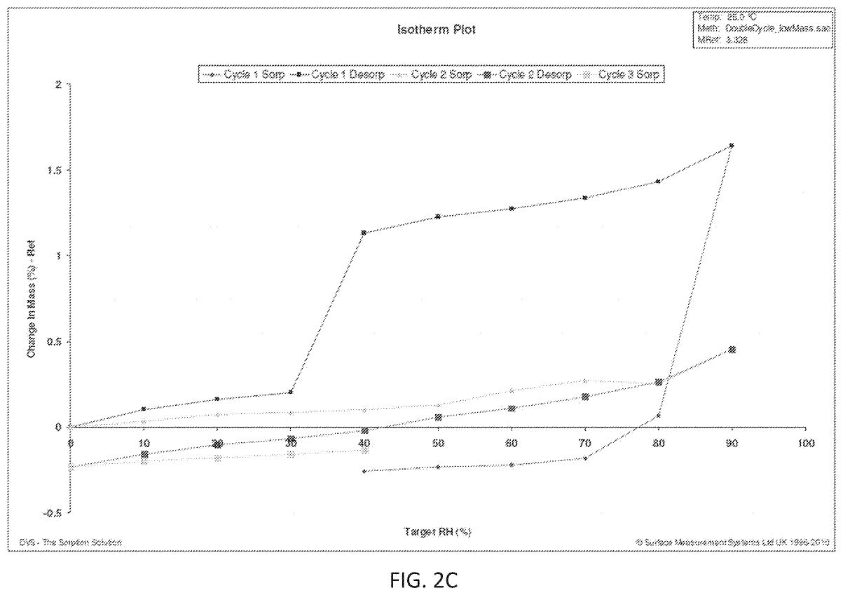

In some embodiments, Form II has an XRPD pattern substantially as shown in FIG. 2A or FIG. 12. In some embodiments, Form II has an XRPD pattern substantially as shown in FIG. 2A. In some embodiments, Form II has an XRPD pattern substantially as shown in FIG. 12.

Angles 2-theta and relative peak intensities that may be observed for Form II using XRPD are shown in Table 2B.

TABLE 2B

| Angle/2θ | d value/Å | Intensity/% |
|---|---|---|
| 8.9 | 9.9 | 9.3 |
| 11.6 | 7.6 | 64.9 |
| 13.0 | 6.8 | 100.0 |
| 13.2 | 6.7 | 41.3 |
| 16.3 | 5.4 | 26.7 |
| 16.6 | 5.3 | 46.0 |
| 17.4 | 5.1 | 82.1 |
| 17.9 | 4.9 | 14.4 |
| 18.6 | 4.8 | 17.6 |
| 18.9 | 4.7 | 72.7 |
| 19.7 | 4.5 | 15.4 |
| 20.4 | 4.4 | 27.9 |
| 20.7 | 4.3 | 38.5 |
| 21.0 | 4.2 | 28.3 |
| 21.3 | 4.2 | 21.8 |
| 22.3 | 4.0 | 63.0 |
| 22.7 | 3.9 | 21.8 |
| 23.2 | 3.8 | 20.4 |
| 24.3 | 3.7 | 18.2 |
| 25.5 | 3.5 | 50.4 |
| 26.2 | 3.4 | 23.6 |
| 26.6 | 3.4 | 20.4 |
| 27.1 | 3.3 | 39.1 |
| 27.4 | 3.3 | 25.1 |
| 28.1 | 3.2 | 12.1 |
| 28.7 | 3.1 | 19.5 |
| 29.7 | 3.0 | 17.2 |
| 30.6 | 2.9 | 20.0 |

In some embodiments, polymorphic Form II has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 2A or 12 or as provided in Table 2B. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form II, can vary by about ±0.6 degrees, ±0.4 degrees, ±0.2 degrees, or ±0.1 degrees 2-theta.

In some embodiments, polymorphic Form II has an XRPD pattern comprising peaks at angles 2-theta of 8.9±0.2, 11.6±0.2, 13.0±0.2, 13.2±0.2, 16.3±0.2, 16.6±0.2, 17.4±0.2, 17.9±0.2, 18.6±0.2, 18.9±0.2, 19.7±0.2, 20.4±0.2, 20.7±0.2, 21.0±0.2, 21.3±0.2, 22.3±0.2, 22.7±0.2, 23.2±0.2, 24.3±0.2, 25.5±0.2, 26.2±0.2, 26.6±0.2, 27.1±0.2, 27.4±0.2, 28.1±0.2, 28.7±0.2, 29.7±0.2, and 30.6±0.2 degrees. In some embodiments, polymorphic Form II has an XRPD pattern comprising peaks at angles 2-theta of 11.6±0.2, 13.0±0.2, 13.2±0.2, 16.6±0.2, 17.4±0.2, 18.9±0.2, 20.7±0.2, 22.3±0.2, 25.5±0.2, and 27.1±0.2 degrees. In some embodiments, polymorphic Form II has an XRPD pattern comprising peaks at angles 2-theta of 11.6±0.2, 13.0±0.2, 17.4±0.2, 18.9±0.2, and 22.3±0.2 degrees. It is to be understood that additional peaks in the XRPD pattern other than those shown in FIG. 2A or 12 or as provided in Table 2B may be observed, for instance, due to the presence of impurities, solvent, or other polymorphs or amorphic forms present in the test sample.

In some embodiments, Form II has a DSC graph substantially as shown in FIG. 2B. In some embodiments, Form II is characterized as having a melting endotherm onset at about 191° C. as determined by DSC. In some embodiments, Form II is characterized as having a melting endotherm onset at about 191±2° C. (e.g., 191±1.9° C., 191±1.8° C., 191±1.7° C., 191±1.6° C., 191±1.5° C., 191±1.4° C., 191±1.3° C., 191±1.2° C., 191±1, 191±0.9° C., 191±0.8° C., 191±0.7° C., 191±0.6° C., 191±0.5° C., 191±0.4° C., 191±0.3° C., 191±0.2° C., or 191±0.1° C.) as determined by DSC.

In some embodiments, Form II has a TGA graph substantially as shown in FIG. 2B.

In some embodiments, Form II has a GVS graph substantially as shown in FIG. 2C.

In some embodiments of Form II, at least one, at least two, at least three, at least four, at least five, or all of the following (a)-(f) apply:

(a) Form II has an XRPD pattern comprising peaks at angles 2-theta of 11.6±0.2, 13.0±0.2, 17.4±0.2, 18.9±0.2, and 22.3±0.2 degrees; an XRPD pattern comprising peaks at angles 2-theta of 11.6±0.2, 13.0±0.2, 13.2±0.2, 16.6±0.2, 17.4±0.2, 18.9±0.2, 20.7±0.2, 22.3±0.2, 25.5±0.2, and 27.1±0.2 degrees; or an XRPD pattern comprising peaks at angles 2-theta of 8.9±0.2, 11.6±0.2, 13.0±0.2, 13.2±0.2, 16.3±0.2, 16.6±0.2, 17.4±0.2, 17.9±0.2, 18.6±0.2, 18.9±0.2, 19.7±0.2, 20.4±0.2, 20.7±0.2, 21.0±0.2, 21.3±0.2, 22.3±0.2, 22.7±0.2, 23.2±0.2, 24.3±0.2, 25.5±0.2, 26.2±0.2, 26.6±0.2, 27.1±0.2, 27.4±0.2, 28.1±0.2, 28.7±0.2, 29.7±0.2, and 30.6±0.2 degrees;
(b) Form II has an XRPD pattern substantially as shown in FIG. 2A or FIG. 12;
(c) Form II has a DSC graph substantially as shown in FIG. 2B;
(d) Form II is characterized as having a melting endotherm onset at about 191° C. as determined by DSC;
(e) Form II has a TGA graph substantially as shown in FIG. 2B; and
(f) Form II has a GVS graph substantially as shown in FIG. 2C.

### Form III

In some embodiments, provided herein is polymorphic Form III of 1-(2-((((trans)-3-fluoro-1-(3-fluoropyridin-2-yl)cyclobutyl)methyl)amino)pyrimidin-5-yl)-1H-pyrrole-3-carboxamide dioxane solvate. Crystal structure information of Form III is provided in Table 3A.

TABLE 3A

| Molecular | C₂₅H₃₀F₂N₆O₄ | | | | |
|---|---|---|---|---|---|
| formula | | | | | |
| Molecular weight | 516.55 | | | | |
| Crystal system | Triclinic | | | | |
| Space group | P-1 | a | 10.1208(7) Å, | α | 89.949(5)°, |
| | | b | 10.6527(6) Å, | β | 77.005(5)°, |
| | | c | 11.9606(7) Å, | γ | 82.186(5)° |

| | |
|---|---|
| V | 1244.24(13) Å³ |
| Z | 2 |
| D_{c} | 1.379 g · cm⁻¹ |
| μ | 0.887 mm⁻¹ |
| Source, λ | Cu—K(alpha), 1.54178 Å |
| F(000) | 544 |
| T | 100(1)K |
| Crystal | Prism colourless, 0.2 × 0.2 × 0.08 mm |
| Data truncated to | 0.80 Å |
| θₘₐₓ | 73.51° |
| Completeness | 96.8% |
| Reflections | 8961 |
| Unique reflections | 4849 |
| Rᵢₙₜ | 0.0193 |

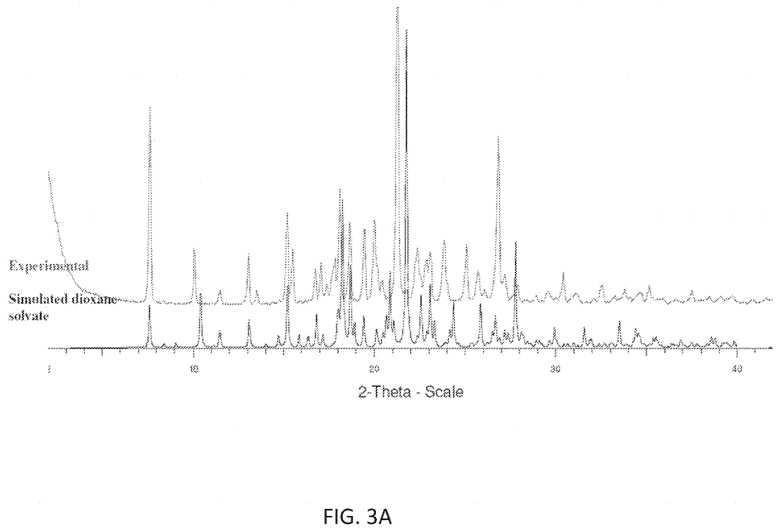

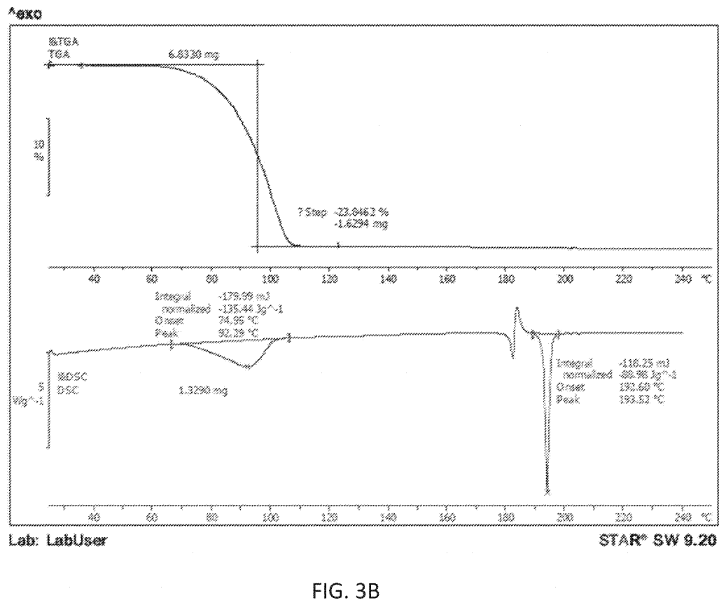

In some embodiments, Form III has an XRPD pattern substantially as shown in FIG. 3A or FIG. 12. In some embodiments, Form III has an XRPD pattern substantially as shown in FIG. 3A. In some embodiments, Form III has an XRPD pattern substantially as shown in FIG. 12.

Angles 2-theta and relative peak intensities that may be observed for Form III using XRPD are shown in Table 3B.

TABLE 3B

| Angle/2θ | d value/Å | Intensity/% |
|---|---|---|
| 7.6 | 11.6 | 64.7 |
| 10.1 | 8.8 | 19.0 |
| 11.5 | 7.7 | 5.9 |
| 13.0 | 6.8 | 17.4 |
| 13.5 | 6.5 | 5.8 |
| 15.1 | 5.8 | 30.4 |
| 15.5 | 5.7 | 19.1 |
| 16.7 | 5.3 | 12.7 |
| 17.1 | 5.2 | 14.6 |
| 17.4 | 5.1 | 8.0 |
| 17.8 | 5.0 | 16.0 |
| 18.1 | 4.9 | 38.2 |
| 18.6 | 4.8 | 27.6 |
| 19.4 | 4.6 | 25.5 |
| 20.0 | 4.4 | 28.3 |
| 20.5 | 4.3 | 9.3 |
| 21.3 | 4.2 | 100.0 |
| 21.7 | 4.1 | 6.4 |
| 22.4 | 4.0 | 19.7 |
| 22.8 | 3.9 | 16.3 |
| 23.0 | 3.9 | 18.0 |
| 23.8 | 3.7 | 22.0 |
| 25.1 | 3.6 | 20.4 |
| 25.7 | 3.5 | 12.3 |
| 26.1 | 3.4 | 6.0 |
| 26.8 | 3.3 | 54.9 |
| 27.2 | 3.3 | 11.0 |
| 27.9 | 3.2 | 7.7 |
| 28.9 | 3.1 | 4.6 |
| 29.6 | 3.0 | 5.7 |
| 30.4 | 2.9 | 11.7 |
| 31.1 | 2.9 | 5.1 |
| 32.6 | 2.7 | 7.7 |

In some embodiments, polymorphic Form III has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 3A or 12 or as provided in Table 3B. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form III, can vary by about ±0.6 degrees, ±0.4 degrees, ±0.2 degrees, or ±0.1 degrees 2-theta.

In some embodiments, polymorphic Form III has an XRPD pattern comprising peaks at angles 2-theta of 7.6±0.2, 10.1±0.2, 11.5±0.2, 13.0±0.2, 13.5±0.2, 15.1±0.2, 15.5±0.2, 16.7±0.2, 17.1±0.2, 17.4±0.2, 17.8±0.2, 18.1±0.2, 18.6±0.2, 19.4±0.2, 20.0±0.2, 20.5±0.2, 21.3±0.2, 21.7±0.2, 22.4±0.2, 22.8±0.2, 23.0±0.2, 23.8±0.2, 25.1±0.2, 25.7±0.2, 26.1±0.2, 26.8±0.2, 27.2±0.2, 27.9±0.2, 28.9±0.2, 29.6±0.2, 30.4±0.2, 31.1±0.2, and 32.6±0.2 degrees. In some embodiments, polymorphic Form III has an XRPD pattern comprising peaks at angles 2-theta of 7.6±0.2, 15.1±0.2, 18.1±0.2, 18.6±0.2, 19.4±0.2, 20.0±0.2, 21.3±0.2, 23.8±0.2, 25.1±0.2, and 26.8±0.2 degrees. In some embodiments, polymorphic Form III has an XRPD pattern comprising peaks at angles 2-theta of 7.6±0.2, 15.1±0.2, 18.1±0.2, 21.3±0.2, and 26.8±0.2 degrees. It is to be understood that additional peaks in the XRPD pattern other than those shown in FIG. 3A or 12 or as provided in Table 3B may be observed, for instance, due to the presence of impurities, solvent, or other polymorphs or amorphic forms present in the test sample.

In some embodiments, Form III has a DSC graph substantially as shown in FIG. 3B. In some embodiments, Form III is characterized as having a broad endotherm with onset at about 75° C. as determined by DSC. In some embodiments, Form III is characterized as having a broad endotherm with onset at 75±2° C. (e.g., 75±1.9° C., 75±1.8° C., 75±1.7° C., 75±1.6° C., 75±1.5° C., 75±1.4° C., 75±1.3° C., 75±1.2° C., 75±1, 75±0.9° C., 75±0.8° C., 75±0.7° C., 75±0.6° C., 75±0.5° C., 75±0.4° C., 75±0.3° C., 75±0.2° C., or 75±0.1° C.) as determined by DSC. In some embodiments, Form III is characterized as having a melting endotherm onset at about 193° C. as determined by DSC. In some embodiments, Form III is characterized as having a melting endotherm onset at 193±2° C. (e.g., 193±1.9° C., 193±1.8° C., 193±1.7° C., 193±1.6° C., 193±1.5° C., 193±1.4° C., 193±1.3° C., 193±1.2° C., 193±1, 193±0.9° C., 193±0.8° C., 193±0.7° C., 193±0.6° C., 193±0.5° C., 193±0.4° C., 193±0.3° C., 193±0.2° C., or 193±0.1° C.) as determined by DSC. In some embodiments, Form III is characterized as having a broad endotherm with onset at about 75° C. and/or a melting endotherm onset at about 193° C. as determined by DSC.

In some embodiments, Form III has a TGA graph substantially as shown in FIG. 3B. In some embodiments, Form III has a weight loss of about 23.8% w/w below 120° C. as determined by TGA.

In some embodiments of Form III, at least one, at least two, at least three, at least four, at least five, or all of the following (a)-(f) apply:

(a) Form III has an XRPD pattern comprising peaks at angles 2-theta of 7.6±0.2, 15.1±0.2, 18.1±0.2, 21.3±0.2, and 26.8±0.2 degrees; an XRPD pattern comprising peaks at angles 2-theta of 7.6±0.2, 15.1±0.2, 18.1±0.2, 18.6±0.2, 19.4±0.2, 20.0±0.2, 21.3±0.2, 23.8±0.2, 25.1±0.2, and 26.8±0.2 degrees; or an XRPD pattern comprising peaks at angles 2-theta of 7.6±0.2, 10.1±0.2, 11.5±0.2, 13.0±0.2, 13.5±0.2, 15.1±0.2, 15.5±0.2, 16.7±0.2, 17.1±0.2, 17.4±0.2, 17.8±0.2, 18.1±0.2, 18.6±0.2, 19.4±0.2, 20.0±0.2, 20.5±0.2, 21.3±0.2, 21.7±0.2, 22.4±0.2, 22.8±0.2, 23.0±0.2, 23.8±0.2, 25.1±0.2, 25.7±0.2, 26.1±0.2, 26.8±0.2, 27.2±0.2, 27.9±0.2, 28.9±0.2, 29.6±0.2, 30.4±0.2, 31.1±0.2, and 32.6±0.2 degrees;
(b) Form III has an XRPD pattern substantially as shown in FIG. 3A or FIG. 12;
(c) Form III has a DSC graph substantially as shown in FIG. 3B;
(d) Form III is characterized as having a broad endotherm with onset at about 75° C. and/or a melting endotherm onset at about 193° C. as determined by DSC;
(e) Form III has a TGA graph substantially as shown in FIG. 3B; and
(f) Form III has a weight loss of about 23.8% w/w below 120° C. as determined by TGA.

### Form IV

In some embodiments, provided herein is polymorphic Form IV of 1-(2-((((trans)-3-fluoro-1-(3-fluoropyridin-2-yl)cyclobutyl)methyl)amino)pyrimidin-5-yl)-1H-pyrrole-3-carboxamide.

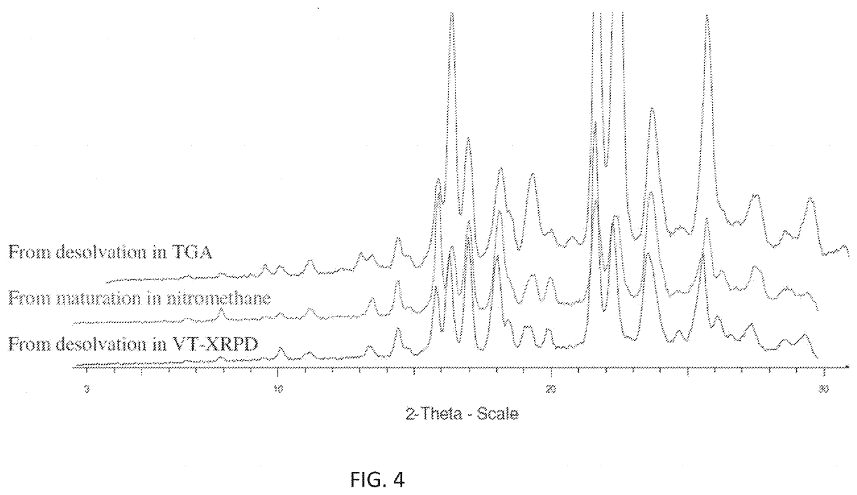

In some embodiments, Form IV has an XRPD pattern substantially as shown in FIG. 4 or FIG. 12. In some embodiments, Form IV has an XRPD pattern substantially as shown in FIG. 4. In some embodiments, Form IV has an XRPD pattern substantially as shown in FIG. 12.

Angles 2-theta and relative peak intensities that may be observed for Form IV using XRPD are shown in Table 4.

TABLE 4

| Angle/2θ | d value/Å | Intensity/% |
|---|---|---|
| 7.9 | 11.2 | 10.1 |
| 9.5 | 9.3 | 7.9 |
| 10.1 | 8.8 | 9.1 |
| 11.2 | 7.9 | 9.4 |
| 13.0 | 6.8 | 12.8 |
| 13.5 | 6.6 | 10.9 |
| 14.4 | 6.2 | 20.9 |
| 14.8 | 6.0 | 7.3 |
| 15.8 | 5.6 | 44.9 |
| 16.4 | 5.4 | 60.9 |
| 17.0 | 5.2 | 50.8 |
| 18.1 | 4.9 | 53.6 |
| 18.6 | 4.8 | 19.6 |
| 19.2 | 4.6 | 14.9 |
| 19.3 | 4.6 | 17.4 |
| 19.7 | 4.5 | 10.9 |
| 19.9 | 4.4 | 15.6 |
| 20.8 | 4.3 | 9.7 |
| 21.8 | 4.1 | 100.0 |
| 22.1 | 4.0 | 12.8 |
| 22.4 | 4.0 | 71.4 |
| 23.8 | 3.7 | 43.5 |
| 24.0 | 3.7 | 15.8 |
| 24.8 | 3.6 | 8.7 |
| 25.5 | 3.5 | 10.9 |
| 25.8 | 3.4 | 48.6 |
| 26.3 | 3.4 | 14.9 |
| 26.8 | 3.3 | 12.5 |
| 27.6 | 3.2 | 17.2 |
| 28.6 | 3.1 | 9.7 |
| 29.6 | 3.0 | 15.5 |
| 30.9 | 2.9 | 9.0 |
| 31.7 | 2.8 | 28.0 |

In some embodiments, polymorphic Form IV has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 4 or 12 or as provided in Table 4. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form IV, can vary by about ±0.6 degrees, ±0.4 degrees, ±0.2 degrees, or ±0.1 degrees 2-theta.

In some embodiments, polymorphic Form IV has an XRPD pattern comprising peaks at angles 2-theta of 7.9±0.2, 9.5±0.2, 10.1±0.2, 11.2±0.2, 13.0±0.2, 13.5±0.2, 14.4±0.2, 14.8±0.2, 15.8±0.2, 16.4±0.2, 17.0±0.2, 18.1±0.2, 18.6±0.2, 19.2±0.2, 19.3±0.2, 19.7±0.2, 19.9±0.2, 20.8±0.2, 21.8±0.2, 22.1±0.2, 22.4±0.2, 23.8±0.2, 24.0±0.2, 24.8±0.2, 25.5±0.2, 25.8±0.2, 26.3±0.2, 26.8±0.2, 27.6±0.2, 28.6±0.2, 29.6±0.2, 30.9±0.2, and 31.7±0.2 degrees. In some embodiments, polymorphic Form IV has an XRPD pattern comprising peaks at angles 2-theta of 14.4±0.2, 16.4±0.2, 17.0±0.2, 18.1±0.2, 18.6±0.2, 21.8±0.2, 22.4±0.2, 23.8±0.2, 25.8±0.2, and 31.7±0.2 degrees. In some embodiments, polymorphic Form IV has an XRPD pattern comprising peaks at angles 2-theta of 16.4±0.2, 17.0±0.2, 18.1±0.2, 21.8±0.2, and 22.4±0.2 degrees. It is to be understood that additional peaks in the XRPD pattern other than those shown in FIG. 4 or 12 or as provided in Table 4 may be observed, for instance, due to the presence of impurities, solvent, or other polymorphs or amorphic forms present in the test sample.

In some embodiments of Form IV, one or both of the following (a)-(b) apply:

(a) Form IV has an XRPD pattern comprising peaks at angles 2-theta of 16.4±0.2, 17.0±0.2, 18.1±0.2, 21.8±0.2, and 22.4±0.2 degrees; an XRPD pattern comprising peaks at angles 2-theta of 14.4±0.2, 16.4±0.2, 17.0±0.2, 18.1±0.2, 18.6±0.2, 21.8±0.2, 22.4±0.2, 23.8±0.2, 25.8±0.2, and 31.7±0.2 degrees; or an XRPD pattern comprising peaks at angles 2-theta of 7.9±0.2, 9.5±0.2, 10.1±0.2, 11.2±0.2, 13.0±0.2, 13.5±0.2, 14.4±0.2, 14.8±0.2, 15.8±0.2, 16.4±0.2, 17.0±0.2, 18.1±0.2, 18.6±0.2, 19.2±0.2, 19.3±0.2, 19.7±0.2, 19.9±0.2, 20.8±0.2, 21.8±0.2, 22.1±0.2, 22.4±0.2, 23.8±0.2, 24.0±0.2, 24.8±0.2, 25.5±0.2, 25.8±0.2, 26.3±0.2, 26.8±0.2, 27.6±0.2, 28.6±0.2, 29.6±0.2, 30.9±0.2, and 31.7±0.2 degrees; and
(b) Form IV has an XRPD pattern substantially as shown in FIG. 4 or FIG. 12.

### Form V

In some embodiments, provided herein is polymorphic Form V of 1-(2-((((trans)-3-fluoro-1-(3-fluoropyridin-2-yl)cyclobutyl)methyl)amino)pyrimidin-5-yl)-1H-pyrrole-3-carboxamide. Crystal structure information of Form V is provided in Table 5A.

TABLE 5A

| Molecular | C₁₉H₁₈F₂N₆O | | | | |
|---|---|---|---|---|---|
| formula | | | | | |
| Molecular weight | 384.39 | | | | |
| Crystal system | Orthorhombic | | | | |
| Space group | Pbca | a | 7.2746(3) Å, | α | 90°, |
| | | b | 16.2395(5) Å, | β | 90°, |
| | | c | 29.6326(10) Å, | γ | 90° |

| | |
|---|---|
| V | 3500.7(2) Å³ |
| Z | 8 |
| D_{c} | 1.459 g · cm⁻¹ |
| μ | 0.927 mm⁻¹ |
| Source, λ | Mo—K(α), 1.54178 Å |
| F(000) | 1600 |
| T | 100(1)K |
| Crystal | Colourless plate, 0.4 × 0.1 × 0.03 mm |
| Data truncated to | 0.80 Å |
| θₘₐₓ | 74.48° |
| Completeness | 99.6% |
| Reflections | 17371 |
| Unique reflections | 3554 |
| Rᵢₙₜ | 0.0455 |

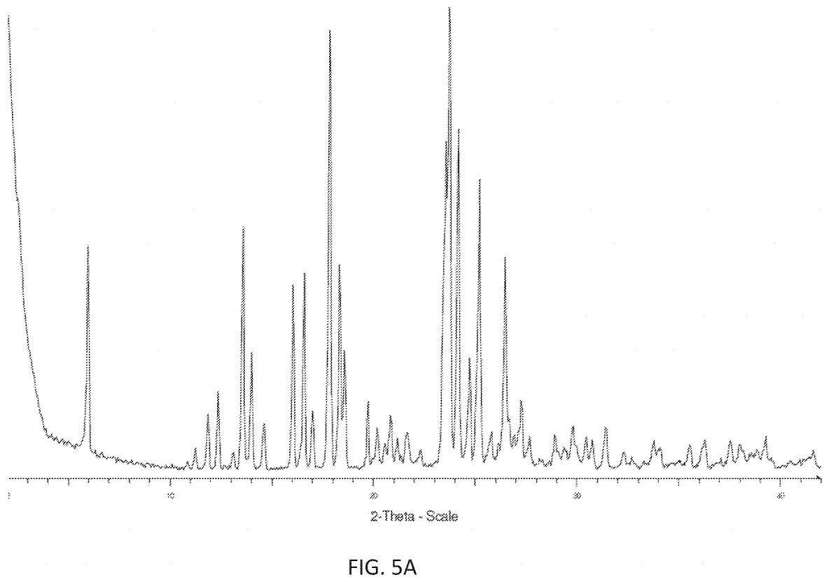

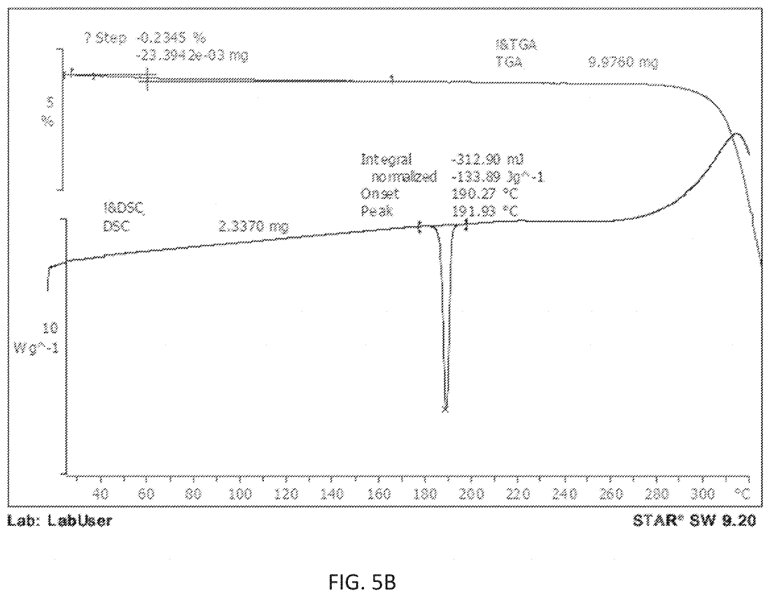

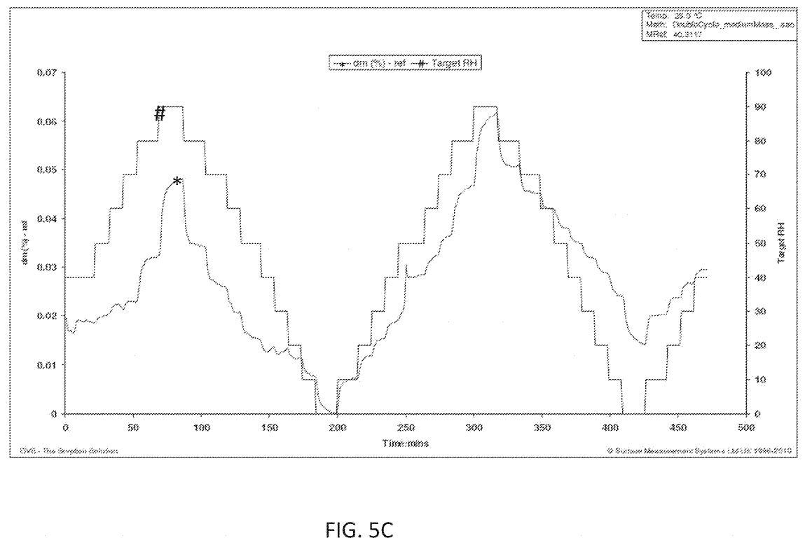

In some embodiments, Form V has an XRPD pattern substantially as shown in FIG. 5A or FIG. 12. In some embodiments, Form V has an XRPD pattern substantially as shown in FIG. 5A. In some embodiments, Form V has an XRPD pattern substantially as shown in FIG. 12.

Angles 2-theta and relative peak intensities that may be observed for Form V using XRPD are shown in Table 5B.

TABLE 5B

| Angle/2θ | d value/Å | Intensity/% |
|---|---|---|
| 5.9 | 14.9 | 48.3 |
| 10.8 | 8.2 | 3.8 |
| 11.2 | 7.9 | 6.1 |
| 11.8 | 7.5 | 13.2 |
| 12.3 | 7.2 | 17.9 |
| 13.0 | 6.8 | 5.3 |
| 13.6 | 6.5 | 52.2 |
| 14.0 | 6.3 | 26.2 |
| 14.6 | 6.1 | 11.1 |
| 16.0 | 5.5 | 40.2 |
| 16.6 | 5.3 | 42.6 |
| 17.0 | 5.2 | 13.8 |
| 17.8 | 5.0 | 93.4 |
| 18.4 | 4.8 | 44.9 |
| 18.6 | 4.8 | 26.8 |
| 19.7 | 4.5 | 15.9 |
| 20.2 | 4.4 | 10.3 |
| 20.6 | 4.3 | 7.3 |
| 20.8 | 4.3 | 13.0 |
| 21.1 | 4.2 | 8.1 |
| 21.7 | 4.1 | 9.4 |
| 22.3 | 4.0 | 5.8 |
| 23.6 | 3.8 | 70.6 |
| 23.7 | 3.8 | 100.0 |
| 24.2 | 3.7 | 72.8 |
| 24.7 | 3.6 | 24.9 |
| 25.2 | 3.5 | 62.2 |
| 25.8 | 3.5 | 9.5 |
| 26.1 | 3.4 | 7.6 |
| 26.5 | 3.4 | 45.9 |
| 26.9 | 3.3 | 9.0 |
| 27.2 | 3.3 | 16.1 |
| 27.7 | 3.2 | 8.0 |
| 29.0 | 3.1 | 8.9 |
| 29.4 | 3.0 | 6.6 |
| 29.9 | 3.0 | 10.8 |
| 30.5 | 2.9 | 8.4 |
| 30.8 | 2.9 | 7.4 |
| 31.4 | 2.8 | 10.5 |
| 32.3 | 2.8 | 5.3 |

In some embodiments, polymorphic Form V has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 5A or 12 or as provided in Table 5B. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form V, can vary by about ±0.6 degrees, ±0.4 degrees, ±0.2 degrees, or ±0.1 degrees 2-theta.

In some embodiments, polymorphic Form V has an XRPD pattern comprising peaks at angles 2-theta of 5.9±0.2, 10.8±0.2, 11.2±0.2, 11.8±0.2, 12.3±0.2, 13.0±0.2, 13.6±0.2, 14.0±0.2, 14.6±0.2, 16.0±0.2, 16.6±0.2, 17.0±0.2, 17.8±0.2, 18.4±0.2, 18.6±0.2, 19.7±0.2, 20.2±0.2, 20.6±0.2, 20.8±0.2, 21.1±0.2, 21.7±0.2, 22.3±0.2, 23.6±0.2, 23.7±0.2, 24.2±0.2, 24.7±0.2, 25.2±0.2, 25.8±0.2, 26.1±0.2, 26.5±0.2, 26.9±0.2, 27.2±0.2, 27.7±0.2, 29.0±0.2, 29.4±0.2, 29.9±0.2, 30.5±0.2, 30.8±0.2, 31.4±0.2, and 32.3±0.2 degrees. In some embodiments, polymorphic Form V has an XRPD pattern comprising peaks at angles 2-theta of 5.9±0.2, 13.6±0.2, 16.6±0.2, 17.8±0.2, 18.4±0.2, 23.6±0.2, 23.7±0.2, 24.2±0.2, 25.2±0.2, and 26.5±0.2 degrees. In some embodiments, polymorphic Form V has an XRPD pattern comprising peaks at angles 2-theta of 17.8±0.2, 23.6±0.2, 23.7±0.2, 24.2±0.2, and 25.2±0.2 degrees. It is to be understood that additional peaks in the XRPD pattern other than those shown in FIG. 5A or 12 or as provided in Table 5B may be observed, for instance, due to the presence of impurities, solvent, or other polymorphs or amorphic forms present in the test sample.

In some embodiments, Form V has a DSC graph substantially as shown in FIG. 5B. In some embodiments, Form V is characterized as having a melting endotherm onset at about 190° C. In some embodiments, Form V is characterized as having a melting endotherm onset at about 190±2° C. (e.g., 190±1.9° C., 190±1.8° C., 190±1.7° C., 190±1.6° C., 190±1.5° C., 190±1.4° C., 190±1.3° C., 190±1.2° C., 190±1, 190±0.9° C., 190±0.8° C., 190±0.7° C., 190±0.6° C., 190±0.5° C., 190±0.4° C., 190±0.3° C., 190±0.2° C., or 190±0.1° C.) as determined by DSC.

In some embodiments, Form V has a TGA graph substantially as shown in FIG. 5B.

In some embodiments, Form V has a GVS graph substantially as shown in FIG. 5C.

In some embodiments of Form V, at least one, at least two, at least three, at least four, at least five, or all of the following (a)-(f) apply:

(a) Form V has an XRPD pattern comprising peaks at angles 2-theta of 17.8±0.2, 23.6±0.2, 23.7±0.2, 24.2±0.2, and 25.2±0.2 degrees; an XRPD pattern comprising peaks at angles 2-theta of 5.9±0.2, 13.6±0.2, 16.6±0.2, 17.8±0.2, 18.4±0.2, 23.6±0.2, 23.7±0.2, 24.2±0.2, 25.2±0.2, and 26.5±0.2 degrees; or an XRPD pattern comprising peaks at angles 2-theta of 5.9±0.2, 10.8±0.2, 11.2±0.2, 11.8±0.2, 12.3±0.2, 13.0±0.2, 13.6±0.2, 14.0±0.2, 14.6±0.2, 16.0±0.2, 16.6±0.2, 17.0±0.2, 17.8±0.2, 18.4±0.2, 18.6±0.2, 19.7±0.2, 20.2±0.2, 20.6±0.2, 20.8±0.2, 21.1±0.2, 21.7±0.2, 22.3±0.2, 23.6±0.2, 23.7±0.2, 24.2±0.2, 24.7±0.2, 25.2±0.2, 25.8±0.2, 26.1±0.2, 26.5±0.2, 26.9±0.2, 27.2±0.2, 27.7±0.2, 29.0±0.2, 29.4±0.2, 29.9±0.2, 30.5±0.2, 30.8±0.2, 31.4±0.2, and 32.3±0.2 degrees;
(b) Form V has an XRPD pattern substantially as shown in FIG. 5A;
(c) Form V has a DSC graph substantially as shown in FIG. 5B;
(d) Form V is characterized as having a melting endotherm onset at about 190° C. as determined by DSC;
(e) Form V has a TGA graph substantially as shown in FIG. 5B; and
(f) Form V has a GVS graph substantially as shown in FIG. 5C.

### Compositions

Also provided herein are compositions containing polymorphs described herein, such as Form I, Form II, Form III, Form IV, Form V, or a mixture thereof. In some embodiments, the composition contains Form I. In some embodiments, the composition contains Form II. In some embodiments, the composition contains Form III. In some embodiments, the composition contains Form IV. In some embodiments, the composition contains Form V. In some embodiment, the composition further comprises a pharmaceutically acceptable carrier.

In some embodiments, provided is a composition containing Form I of 1-(2-((((trans)-3-fluoro-1-(3-fluoropyridin-2-yl)cyclobutyl)methyl)amino)pyrimidin-5-yl)-1H-pyrrole-3-carboxamide. In some embodiments, the composition is substantially free of at least one, at least two, at least three, or all of polymorphic Forms II-V of 1-(2-((((trans)-3-fluoro-1-(3-fluoropyridin-2-yl)cyclobutyl)methyl)amino)pyrimidin-5-yl)-1H-pyrrole-3-carboxamide. In some embodiments, the composition is substantially free of amorphous or non-crystalline form of 1-(2-((((trans)-3-fluoro-1-(3-fluoropyridin-2-yl)cyclobutyl)methyl)amino)pyrimidin-5-yl)-1H-pyrrole-3-carboxamide. In some embodiments, the composition is substantially free of salts of 1-(2-((((trans)-3-fluoro-1-(3-fluoropyridin-2-yl)cyclobutyl)methyl)amino)pyrimidin-5-yl)-1H-pyrrole-3-carboxamide.

In some embodiments of the composition containing Form I of 1-(2-((((trans)-3-fluoro-1-(3-fluoropyridin-2-yl)cyclobutyl)methyl)amino)pyrimidin-5-yl)-1H-pyrrole-3-carboxamide, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, or at least about 99% by weight of the total composition is Form I.

In some embodiments, provided is a composition containing Form II of 1-(2-((((trans)-3-fluoro-1-(3-fluoropyridin-2-yl)cyclobutyl)methyl)amino)pyrimidin-5-yl)-1H-pyrrole-3-carboxamide. In some embodiments, the composition is substantially free of at least one, at least two, at least three, or all of polymorphic Forms I and III-V of 1-(2-((((trans)-3-fluoro-1-(3-fluoropyridin-2-yl)cyclobutyl)methyl)amino)pyrimidin-5-yl)-1H-pyrrole-3-carboxamide. In some embodiments, the composition is substantially free of amorphous or non-crystalline form of 1-(2-((((trans)-3-fluoro-1-(3-fluoropyridin-2-yl)cyclobutyl)methyl)amino)pyrimidin-5-yl)-1H-pyrrole-3-carboxamide. In some embodiments, the composition is substantially free of salts of 1-(2-((((trans)-3-fluoro-1-(3-fluoropyridin-2-yl)cyclobutyl)methyl)amino)pyrimidin-5-yl)-1H-pyrrole-3-carboxamide.

In some embodiments of the composition containing Form II of 1-(2-((((trans)-3-fluoro-1-(3-fluoropyridin-2-yl)cyclobutyl)methyl)amino)pyrimidin-5-yl)-1H-pyrrole-3-carboxamide, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, or at least about 99% by weight of the total composition is Form II.

In some embodiments, provided is a composition containing Form III of 1-(2-((((trans)-3-fluoro-1-(3-fluoropyridin-2-yl)cyclobutyl)methyl)amino)pyrimidin-5-yl)-1H-pyrrole-3-carboxamide. In some embodiments, the composition is substantially free of at least one, at least two, at least three, or all of polymorphic Forms I, II, IV and V of 1-(2-((((trans)-3-fluoro-1-(3-fluoropyridin-2-yl)cyclobutyl)methyl)amino)pyrimidin-5-yl)-1H-pyrrole-3-carboxamide. In some embodiments, the composition is substantially free of amorphous or non-crystalline form of 1-(2-((((trans)-3-fluoro-1-(3-fluoropyridin-2-yl)cyclobutyl)methyl)amino)pyrimidin-5-yl)-1H-pyrrole-3-carboxamide. In some embodiments, the composition is substantially free of salts of 1-(2-((((trans)-3-fluoro-1-(3-fluoropyridin-2-yl)cyclobutyl)methyl)amino)pyrimidin-5-yl)-1H-pyrrole-3-carboxamide.

In some embodiments of the composition containing Form III of 1-(2-((((trans)-3-fluoro-1-(3-fluoropyridin-2-yl)cyclobutyl)methyl)amino)pyrimidin-5-yl)-1H-pyrrole-3-carboxamide, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, or at least about 99% by weight of the total composition is Form III.

In some embodiments, provided is a composition containing Form IV of 1-(2-((((trans)-3-fluoro-1-(3-fluoropyridin-2-yl)cyclobutyl)methyl)amino)pyrimidin-5-yl)-1H-pyrrole-3-carboxamide. In some embodiments, the composition is substantially free of at least one, at least two, at least three, or all of polymorphic Forms I-III and V of 1-(2-((((trans)-3-fluoro-1-(3-fluoropyridin-2-yl)cyclobutyl)methyl)amino)pyrimidin-5-yl)-1H-pyrrole-3-carboxamide. In some embodiments, the composition is substantially free of amorphous or non-crystalline form of 1-(2-((((trans)-3-fluoro-1-(3-fluoropyridin-2-yl)cyclobutyl)methyl)amino)pyrimidin-5-yl)-1H-pyrrole-3-carboxamide. In some embodiments, the composition is substantially free of salts of 1-(2-((((trans)-3-fluoro-1-(3-fluoropyridin-2-yl)cyclobutyl)methyl)amino)pyrimidin-5-yl)-1H-pyrrole-3-carboxamide.

In some embodiments of the composition containing Form IV of 1-(2-((((trans)-3-fluoro-1-(3-fluoropyridin-2-yl)cyclobutyl)methyl)amino)pyrimidin-5-yl)-1H-pyrrole-3-carboxamide, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, or at least about 99% by weight of the total composition is Form IV.

In some embodiments, provided is a composition containing Form V of 1-(2-((((trans)-3-fluoro-1-(3-fluoropyridin-2-yl)cyclobutyl)methyl)amino)pyrimidin-5-yl)-1H-pyrrole-3-carboxamide. In some embodiments, the composition is substantially free of at least one, at least two, at least three, or all of polymorphic Forms I-IV of 1-(2-((((trans)-3-fluoro-1-(3-fluoropyridin-2-yl)cyclobutyl)methyl)amino)pyrimidin-5-yl)-1H-pyrrole-3-carboxamide. In some embodiments, the composition is substantially free of amorphous or non-crystalline form of 1-(2-((((trans)-3-fluoro-1-(3-fluoropyridin-2-yl)cyclobutyl)methyl)amino)pyrimidin-5-yl)-1H-pyrrole-3-carboxamide. In some embodiments, the composition is substantially free of salts of 1-(2-((((trans)-3-fluoro-1-(3-fluoropyridin-2-yl)cyclobutyl)methyl)amino)pyrimidin-5-yl)-1H-pyrrole-3-carboxamide.

In some embodiments of the composition containing Form V of 1-(2-((((trans)-3-fluoro-1-(3-fluoropyridin-2-yl)cyclobutyl)methyl)amino)pyrimidin-5-yl)-1H-pyrrole-3-carboxamide, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, or at least about 99% by weight of the total composition is Form V.

In some embodiments, provided is a composition containing Form I and Form V of 1-(2-((((trans)-3-fluoro-1-(3-fluoropyridin-2-yl)cyclobutyl)methyl)amino)pyrimidin-5-yl)-1H-pyrrole-3-carboxamide. In some embodiments, Form I and Form V are present in a weight ratio of 99 to 1, 90 to 10, 80 to 20, 70 to 30, 60 to 40, 50 to 50, 40 to 60, 30 to 70, 20 to 80, 10 to 90, or 1 to 99. In some embodiments, the weight ratio of Form I to Form V is between 90 to 10 and 99 to 1. In some embodiments of a composition containing Form I and Form V, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, or at least about 99% by weight of the total composition is Form I. In some embodiments of a composition containing Form I and Form V, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, or at least about 99% by weight of the total composition is Form V.

In some embodiments, provided is a tablet or capsule containing one or more of the polymorphic forms described herein (e.g., Form I, II, III, IV, V, or a mixture thereof), and one or more pharmaceutically acceptable carriers. In some embodiments, provided is a tablet or capsule containing substantially pure polymorphic Form I of 1-(2-((((trans)-3-fluoro-1-(3-fluoropyridin-2-yl)cyclobutyl)methyl)amino)pyrimidin-5-yl)-1H-pyrrole-3-carboxamide, and one or more pharmaceutically acceptable carriers. In some embodiments, provided is a tablet or capsule containing substantially pure polymorphic Form II of 1-(2-((((trans)-3-fluoro-1-(3-fluoropyridin-2-yl)cyclobutyl)methyl)amino)pyrimidin-5-yl)-1H-pyrrole-3-carboxamide, and one or more pharmaceutically acceptable carriers. In some embodiments, provided is a tablet or capsule containing substantially pure polymorphic Form III of 1-(2-((((trans)-3-fluoro-1-(3-fluoropyridin-2-yl)cyclobutyl)methyl)amino)pyrimidin-5-yl)-1H-pyrrole-3-carboxamide, and one or more pharmaceutically acceptable carriers. In some embodiments, provided is a tablet or capsule containing substantially pure polymorphic Form IV of 1-(2-((((trans)-3-fluoro-1-(3-fluoropyridin-2-yl)cyclobutyl)methyl)amino)pyrimidin-5-yl)-1H-pyrrole-3-carboxamide, and one or more pharmaceutically acceptable carriers. In some embodiments, provided is a tablet or capsule containing substantially pure polymorphic Form V of 1-(2-((((trans)-3-fluoro-1-(3-fluoropyridin-2-yl)cyclobutyl)methyl)amino)pyrimidin-5-yl)-1H-pyrrole-3-carboxamide, and one or more pharmaceutically acceptable carriers.

The polymorphic forms and compositions described herein may be combined with one or more additional therapeutic agents. Suitable additional therapeutic agents include, for example, anti-obesity agents, anti-sarcopenia agents, anti-wasting syndrome agents, anti-frailty agents, anti-cachexia agents, anti-muscle spasm agents, agents against post-surgical and post-traumatic muscle weakness, and anti-neuromuscular disease agents.

Suitable additional therapeutic agents include, for example: orlistat, sibramine, diethylpropion, phentermine, benzaphetamine, phendimetrazine, estrogen, estradiol, levonorgestrel, norethindrone acetate, estradiol valerate, ethinyl estradiol, norgestimate, conjugated estrogens, esterified estrogens, medroxyprogesterone acetate, testosterone, insulin-derived growth factor, human growth hormone, edaravone, nusinersen, riluzole, cannabidiol, prednisone, albuterol, non-steroidal anti-inflammatory drugs, and botulinum toxin.

Other suitable additional therapeutic agents include TRH, diethylstilbesterol, theophylline, enkephalins, E series prostaglandins, compounds disclosed in U.S. Pat. No. 3,239,345 (e.g., zeranol), compounds disclosed in U.S. Pat. No. 4,036,979 (e.g., sulbenox), peptides disclosed in U.S. Pat. No. 4,411,890, growth hormone secretagogues such as GHRP-6, GHRP-1 (disclosed in U.S. Pat. No. 4,411,890 and publications WO 89/07110 and WO 89/07111), GHRP-2 (disclosed in WO 93/04081), NN703 (Novo Nordisk), LY444711 (Lilly), MK-677 (Merck), CP424391 (Pfizer) and B-HT920, growth hormone releasing factor and its analogs, growth hormone and its analogs and somatomedins including IGF-1 and IGF-2, alpha-adrenergic agonists, such as clonidine or serotonin 5-HT_{D} agonists, such as sumatriptan, agents which inhibit somatostatin or its release, such as physostigmine, pyridostigmine, parathyroid hormone, PTH(1-34), and bisphosphonates, such as MK-217 (alendronate).

Still other suitable additional therapeutic agents include estrogen, testosterone, selective estrogen receptor modulators, such as tamoxifen or raloxifene, other androgen receptor modulators, such as those disclosed in Edwards, J. P. et. al., Bio. Med. Chem. Let., 9, 1003-1008 (1999) and Hamann, L. G. et. al., J. Med. Chem., 42, 210-212 (1999), and progesterone receptor agonists (“PRA”), such as levonorgestrel, medroxyprogesterone acetate (MPA).

Other suitable additional therapeutic agents include anabolic agents, such as selective androgen receptor modulators (SARMs); antagonists of the activin receptor pathway, such as anti-myostatin antibodies or soluble activin receptor decoys, including ACE-031 (Acceleron Pharmaceuticals, a soluble activin receptor type IIB antagonist), MYO-027/PFE-3446879 (Wyeth/Pfizer, an antibody myostatin inhibitor), AMG-745 (Amgen, a peptibody myostatin inhibitor), and an ActRIIB decoy receptor (see Zhou et al., Cell, 142, 531-543, Aug. 20, 2010); and anabolic steroids.

Still other suitable additional therapeutic agents include aP2 inhibitors, such as those disclosed in U.S. Pat. No. 6,548,529, PPAR gamma antagonists, PPAR delta agonists, beta 3 adrenergic agonists, such as AJ9677 (Takeda/Dainippon), L750355 (Merck), or CP331648 (Pfizer), other beta 3 agonists as disclosed in U.S. Pat. Nos. 5,541,204, 5,770,615, 5,491,134, 5,776,983 and 5,488,064, a lipase inhibitor, such as orlistat or ATL-962 (Alizyme), a serotonin (and dopamine) reuptake inhibitor, such as sibutramine, topiramate (Johnson & Johnson) or axokine (Regeneron), a thyroid receptor beta drug, such as a thyroid receptor ligand as disclosed in WO 97/21993, WO 99/00353, and GB98/284425, and anorectic agents, such as dexamphetamine, phentermine, phenylpropanolamine or mazindol.

Still other suitable additional therapeutic agents include HIV and AIDS therapies, such as indinavir sulfate, saquinavir, saquinavir mesylate, ritonavir, lamivudine, zidovudine, lamivudine/zidovudine combinations, zalcitabine, didanosine, stavudine, and megestrol acetate.

Still other suitable additional therapeutic agents include antiresorptive agents, hormone replacement therapies, vitamin D analogues, elemental calcium and calcium supplements, cathepsin K inhibitors, MMP inhibitors, vitronectin receptor antagonists, Src SH.sub.2 antagonists, vacuolar H⁺-ATPase inhibitors, ipriflavone, fluoride, Tibo lone, pro stanoids, 17-beta hydroxysteroid dehydrogenase inhibitors and Src kinase inhibitors.

The above therapeutic agents, when employed in combination with the polymorphic forms and compositions disclosed herein, may be used, for example, in those amounts indicated in the Physicians' Desk Reference (PDR) or as otherwise determined by one of ordinary skill in the art.

The polymorphic forms and compositions disclosed herein may be administered at a therapeutically effective dosage, e.g., a dosage sufficient to provide treatment for the disease state. While human dosage levels have yet to be optimized for the chemical entities described herein, generally, a daily dose ranges from about 0.05 to 100 mg/kg of body weight; in some embodiments, from about 0.10 to 10.0 mg/kg of body weight, and in some embodiments, from about 0.15 to 1.0 mg/kg of body weight. Thus, for administration to a 70 kg person, in some embodiments, the dosage range would be about from 3.5 to 7000 mg per day; in some embodiments, about from 7.0 to 700.0 mg per day, and in some embodiments, about from 10.0 to 100.0 mg per day. The amount of the chemical entity administered will be dependent, for example, on the subject and disease state being treated, the severity of the affliction, the manner and schedule of administration and the judgment of the prescribing physician. In some embodiments, the dosage is about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, once daily, twice daily or three times daily. In some embodiments, the dosage range is from about 10 mg to about 800 mg, from about 50 mg to about 800 mg, from 100 mg to about 800 mg, from about 200 mg to about 800 mg, from about 300 mg to about 800 mg, from about 400 mg to about 800 mg, from about 500 mg to about 800 mg, from about 600 mg to about 800 mg, from about 700 mg to about 800 mg, from about 10 mg to about 700 mg, from about 50 mg to about 700 mg, from 100 mg to about 700 mg, from about 200 mg to about 700 mg, from about 300 mg to about 700 mg, from about 400 mg to about 700 mg, from about 500 mg to about 700 mg, from about 600 mg to about 700 mg, from about 10 mg to about 600 mg, from about 50 mg to about 600 mg, from 100 mg to about 600 mg, from about 200 mg to about 600 mg, from about 300 mg to about 600 mg, from about 400 mg to about 600 mg, from about 500 mg to about 600 mg, from about 10 mg to about 500 mg, from about 50 mg to about 500 mg, from 100 mg to about 500 mg, from about 200 mg to about 500 mg, from about 300 mg to about 500 mg, from about 400 mg to about 500 mg, from about 10 mg to about 500 mg, from about 50 mg to about 500 mg, from 100 mg to about 500 mg, from about 200 mg to about 500 mg, from about 300 mg to about 500 mg, from about 400 mg to about 500 mg, from about 10 mg to about 400 mg, from about 50 mg to about 400 mg, from 100 mg to about 400 mg, from about 200 mg to about 400 mg, from about 300 mg to about 400 mg, from about 10 mg to about 300 mg, from about 50 mg to about 300 mg, from 100 mg to about 300 mg, from about 200 mg to about 300 mg, from about 10 mg to about 200 mg, from about 50 mg to about 200 mg, from 100 mg to about 200 mg, from about 10 mg to about 100 mg, or from about 50 mg to about 100 mg.

Administration of the polymorphic forms and compositions disclosed herein can be via any accepted mode of administration for therapeutic agents including, but not limited to, oral, sublingual, subcutaneous, parenteral, intravenous, intranasal, topical, transdermal, intraperitoneal, intramuscular, intrapulmonary, vaginal, rectal, or intraocular administration. In some embodiments, the compound or composition is administered orally or intravenously. In some embodiments, the compound or composition disclosed and/or described herein is administered orally.

Pharmaceutically acceptable compositions include solid, semi-solid, liquid and aerosol dosage forms, such as tablet, capsule, powder, liquid, suspension, suppository, and aerosol forms. The compounds disclosed and/or described herein can also be administered in sustained or controlled release dosage forms (e.g., controlled/sustained release pill, depot injection, osmotic pump, or transdermal (including electrotransport) patch forms) for prolonged timed, and/or pulsed administration at a predetermined rate. In some embodiments, the compositions are provided in unit dosage forms suitable for single administration of a precise dose.

The polymorphic forms and compositions disclosed herein can be administered either alone or in combination with one or more conventional pharmaceutically acceptable carriers (e.g., mannitol, lactose, starch, magnesium stearate, sodium saccharine, talcum, cellulose, sodium crosscarmellose, glucose, gelatin, sucrose, magnesium carbonate). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate). Generally, depending on the intended mode of administration, the pharmaceutical composition will contain about 0.005% to 95%, or about 0.5% to 50%, by weight of a compound disclosed and/or described herein. Actual methods of preparing such dosage forms are known, or will be apparent, to those skilled in this art; for example, see Remington's Pharmaceutical Sciences, Mack Publishing Company, Easton, Pa.

The compositions described herein can be manufactured using any conventional method, e.g., mixing, dissolving, granulating, dragee-making, levigating, emulsifying, encapsulating, entrapping, melt-spinning, spray-drying, or lyophilizing processes. An optimal pharmaceutical formulation can be determined by one of skill in the art depending on the route of administration and the desired dosage. Such formulations can influence the physical state, stability, rate of in vivo release, and rate of in vivo clearance of the administered agent. Depending on the condition being treated, these pharmaceutical compositions can be formulated and administered systemically or locally.

Alternatively, formulations for parenteral use can include dispersions or suspensions of polymorphic forms described herein prepared as appropriate oily injection suspensions. Suitable lipophilic solvents or vehicles include fatty oils, such as sesame oil, and synthetic fatty acid esters, such as ethyl oleate or triglycerides, or liposomes. Aqueous injection suspensions can contain substances that increase the viscosity of the suspension, such as sodium carboxymethylcellulose, sorbitol, dextran, and mixtures thereof. Optionally, the suspension also can contain suitable stabilizers or agents that increase the solubility of the compounds to allow for the preparation of highly concentrated solutions. Aqueous polymers that provide pH-sensitive solubilization and/or sustained release of the active agent also can be used as coatings or matrix structures, e.g., methacrylic polymers, such as the EUDRAGIT™ series available from Rohm America Inc. (Piscataway, N.J.). Emulsions, e.g., oil-in-water and water-in-oil dispersions, also can be used, optionally stabilized by an emulsifying agent or dispersant (surface active materials; surfactants). Suspensions can contain suspending agents such as ethoxylated isostearyl alcohols, polyoxyethlyene sorbitol and sorbitan esters, microcrystalline cellulose, aluminum metahydroxide, bentonite, agar-agar, gum tragacanth, and mixtures thereof.

Liposomes containing the polymorphic forms described herein also can be employed for parenteral administration. Liposomes generally are derived from phospholipids or other lipid substances. The compositions in liposome form also can contain other ingredients, such as stabilizers, preservatives, excipients, and the like. Preferred lipids include phospholipids and phosphatidyl cholines (lecithins), both natural and synthetic. Methods of forming liposomes are known in the art. See, e.g., Prescott (Ed.), Methods in Cell Biology, Vol. XIV, p. 33, Academic Press, New York (1976).

In some embodiments, the polymorphic forms or compositions disclosed herein are formulated for oral administration using pharmaceutically acceptable carriers well known in the art. Preparations formulated for oral administration can be in the form of tablets, pills, capsules, cachets, dragees, lozenges, liquids, gels, syrups, slurries, elixirs, suspensions, or powders. To illustrate, pharmaceutical preparations for oral use can be obtained by combining the active compounds with a solid excipient, optionally grinding the resulting mixture, and processing the mixture of granules, after adding suitable auxiliaries if desired, to obtain tablets or dragee cores. Oral formulations can employ liquid carriers similar in type to those described for parenteral use, e.g., buffered aqueous solutions, suspensions, and the like.

Examples of oral formulations include tablets, dragees, and gelatin capsules. These preparations can contain one or more carriers, which include, without limitation:

a) diluents, such as microcrystalline cellulose and sugars, including lactose, dextrose, sucrose, mannitol, or sorbitol;
b) binders, such as sodium starch glycolate, croscarmellose sodium, magnesium aluminum silicate, starch from corn, wheat, rice, potato, etc.;
c) cellulose materials, such as methylcellulose, hydroxypropylmethyl cellulose, and sodium carboxymethylcellulose, polyvinylpyrrolidone, gums, such as gum arabic and gum tragacanth, and proteins, such as gelatin and collagen;
d) disintegrating or solubilizing agents such as cross-linked polyvinyl pyrrolidone, starches, agar, alginic acid or a salt thereof, such as sodium alginate, or effervescent compositions;
e) lubricants, such as silica, talc, stearic acid or its magnesium or calcium salt, and polyethylene glycol;
f) flavorants and sweeteners;
g) colorants or pigments, e.g., to identify the product or to characterize the quantity (dosage) of active compound; and
h) other ingredients, such as preservatives, stabilizers, swelling agents, emulsifying agents, solution promoters, salts for regulating osmotic pressure, and buffers.

### Methods of Preparation

1-(2-((((trans)-3-fluoro-1-(3-fluoropyridin-2-yl)cyclobutyl)methyl)amino)pyrimidin-5-yl)-1H-pyrrole-3-carboxamide can be synthesized by synthetic methods known to the skilled artisan, for example, as described in U.S. Pat. No. 8,962,632 and as described herein.

### Form I

In some embodiments, provided is a method of preparing polymorphic Form I of 1-(2-((((trans)-3-fluoro-1-(3-fluoropyridin-2-yl)cyclobutyl)methyl)amino)pyrimidin-5-yl)-1H-pyrrole-3-carboxamide, the method comprising: (a) mixing 1-(2-((((trans)-3-fluoro-1-(3-fluoropyridin-2-yl)cyclobutyl)methyl)amino)pyrimidin-5-yl)-1H-pyrrole-3-carboxamide with a solvent; and (b) subjecting the mixture generated in step (a) to heat/cool cycles. In some embodiments, the solvent is selected from the group consisting of toluene, anisole, heptane, tert-butyl methyl ether (TBME), methyl isobutyl ketone (MIBK), methyl ethyl ketone (MEK), ethanol, acetonitrile, methanol, butyl acetate (BuOAc), isopropyl acetate (IPAc), 1-butanol, 1-propanol, 2-propanol, methylene dichloride (DCM), water, ethanol/5% water, and isopropyl alcohol (IPA)/5% water. In some embodiments, the heat/cool cycles comprises cycles between room temperature and about 50° C., wherein the duration of each condition is about four hours. In some embodiments, the method further comprises filtering a solid generated in step (b) after 5 days. In some embodiments, the method further comprises filtering a solid generated in step (b) after 20 days.

### Form II

In some embodiments, provided is a method of preparing polymorphic Form II of 1-(2-((((trans)-3-fluoro-1-(3-fluoropyridin-2-yl)cyclobutyl)methyl)amino)pyrimidin-5-yl)-1H-pyrrole-3-carboxamide, the method comprising; (a) mixing polymorphic Form I of 1-(2-((((trans)-3-fluoro-1-(3-fluoropyridin-2-yl)cyclobutyl)methyl)amino)pyrimidin-5-yl)-1H-pyrrole-3-carboxamide with a solvent, wherein the solvent is THF/5% water (v/v); and (b) evaporating the mixture of step (a). In some embodiments, step (a) is conducted at a temperature of about 50° C. In some embodiments, the method further comprises filtering a solid generated in step (b).

### Form III

In some embodiments, provided is a method of preparing polymorphic Form III of 1-(2-((((trans)-3-fluoro-1-(3-fluoropyridin-2-yl)cyclobutyl)methyl)amino)pyrimidin-5-yl)-1H-pyrrole-3-carboxamide dioxane solvate, the method comprising: (a) mixing polymorphic Form I of 1-(2-((((trans)-3-fluoro-1-(3-fluoropyridin-2-yl)cyclobutyl)methyl)amino)pyrimidin-5-yl)-1H-pyrrole-3-carboxamide with a solvent, wherein the solvent is dioxane/5% water; and (b) evaporating the mixture of step (a). In some embodiments, step (a) is conducted at a temperature of about 50° C. In some embodiments, the method further comprises filtering a solid generated in step (b).

### Form IV

In some embodiments, provided is a method of preparing polymorphic Form IV of 1-(2-((((trans)-3-fluoro-1-(3-fluoropyridin-2-yl)cyclobutyl)methyl)amino)pyrimidin-5-yl)-1H-pyrrole-3-carboxamide, the method comprising: (a) mixing polymorphic Form I of 1-(2-((((trans)-3-fluoro-1-(3-fluoropyridin-2-yl)cyclobutyl)methyl)amino)pyrimidin-5-yl)-1H-pyrrole-3-carboxamide with THF at a temperature of about 40° C., thereby generating a solid; and (b) heating the solid generated in step (a). In some embodiments, the mixture of step (a) is shaken for about 5 days. In some embodiments, step (b) comprises heating the solid generated in step (a) to a temperature of about 120° C.

### Form V

In some embodiments, provided is a method of preparing polymorphic Form V of 1-(2-((((trans)-3-fluoro-1-(3-fluoropyridin-2-yl)cyclobutyl)methyl)amino)pyrimidin-5-yl)-1H-pyrrole-3-carboxamide, the method comprising: (a) mixing polymorphic Form I of 1-(2-((((trans)-3-fluoro-1-(3-fluoropyridin-2-yl)cyclobutyl)methyl)amino)pyrimidin-5-yl)-1H-pyrrole-3-carboxamide with a solvent, wherein the solvent is an aliphatic alcohol or its mixture with water; and (b) slurring the mixture of step (a). In some embodiments, the aliphatic alcohol has a structure of R—OH, wherein R is an alkyl. “Alkyl” as used herein refers to and includes, unless otherwise stated, a saturated linear (i.e., unbranched) or branched univalent hydrocarbon chain or combination thereof, having the number of carbon atoms designated (i.e., C₁₋₁₀ means one to ten carbon atoms). Particular alkyl groups are those having 1 to 20 carbon atoms (a “C₁₋₂₀ alkyl”), having 1 to 10 carbon atoms (a “C₁₋₁₀ alkyl”), having 6 to 10 carbon atoms (a “C₆₋₁₀ alkyl”), having 1 to 6 carbon atoms (a “C₁₋₆ alkyl”), having 2 to 6 carbon atoms (a “C₂₋₆ alkyl”), or having 1 to 4 carbon atoms (a “C₁₋₄ alkyl”). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like. In some embodiments, the solvent is 1-propanol, aqueous 1-propanol, ethanol, denatured ethanol or aqueous denatured ethanol. In some embodiments, the solvent is aqueous 1-propanol. In some embodiments, the solvent is 75% (v/v) 1-propanol/water. In some embodiments, step (b) is conducted at a temperature of less than about 50° C., less than about 40° C., less than about 30° C., less than about 20° C., less than about 10° C., or less than about 5° C. In some embodiments, step (b) is conducted at a temperature of about 50° C., 40° C., 30° C., 20° C., 10° C., 5° C., or 0° C. In some embodiments, step (b) is conducted at a temperature of about 0° C.

### Methods of Use

In another aspect, provided are methods for enhancing fast skeletal muscle efficiency in a patient in need thereof, comprising administering to said patient an effective amount of a polymorphic form or composition described herein that selectively binds the troponin complex of fast skeletal muscle fiber or sarcomere. In some embodiments, the polymorphic form or composition described herein activates fast skeletal muscle fibers or sarcomeres. In some embodiments, administration of a polymorphic form or composition described herein results in an increase in fast skeletal muscle power output. In some embodiments, administration of a polymorphic form or composition described herein results in increased sensitivity of fast skeletal muscle fibers or sarcomeres to calcium ion, as compared to fast skeletal muscle fibers or sarcomeres untreated with the compound. In some embodiments, administration of a polymorphic form or composition described herein results in a lower concentration of calcium ions causing fast skeletal muscle myosin to bind to actin. In some embodiments, administration of a polymorphic form or composition described herein results in the fast skeletal muscle fiber generating force to a greater extent at submaximal levels of muscle activation.

Also provided is a method for sensitizing a fast skeletal muscle fiber to produce force in response to a lower concentration of calcium ion, comprising contacting the fast skeletal muscle fiber with a polymorphic form or composition described herein that selectively binds to troponin complexes in the fast skeletal muscle sarcomere. In some embodiments, contacting the fast skeletal muscle fiber with a polymorphic form or composition described herein results in activation of the fast skeletal muscle fiber at a lower calcium ion concentration than in an untreated fast skeletal muscle fiber. In some embodiments, contacting the fast skeletal muscle fiber with a polymorphic form or composition described herein results in the production of increased force at a lower calcium ion concentration in comparison with an untreated fast skeletal muscle fiber.

Also provided is a method for increasing time to fast skeletal muscle fatigue in a patient in need thereof, comprising contacting fast skeletal muscle fibers with a polymorphic form or composition described herein that selectively binds to the troponin complexes of the fast skeletal muscle fibers. In some embodiments, the compound binds to form ligand-troponin-calcium ion complexes that activate the fast skeletal muscle fibers. In some embodiments, formation of the complexes and/or activation of the fast skeletal muscle fibers results in enhanced force and/or increased time to fatigue as compared to untreated fast skeletal muscle fibers contacted with a similar calcium ion concentration.

The polymorphic forms or compositions described herein are capable of modulating the contractility of the fast skeletal sarcomere in vivo, and can have application in both human and animal disease. Modulation would be desirable in a number of conditions or diseases, including, but not limited to, 1) neuromuscular disorders, such as Amyotrophic Lateral Sclerosis (ALS), Spinal Muscular Atrophy (SMA), peripheral neuropathies and myasthenia gravis; 2) disorders of voluntary muscle, including muscular dystrophies, myopathies and conditions of muscle wasting, such as sarcopenia and cachexia syndromes (e.g., cachexia syndromes caused by diseases such as cancer, heart failure, chronic obstructive pulmonary disease (COPD), and chronic kidney disease/dialysis), and rehabilitation-related deficits, such as those associated with recovery from surgery (e.g. post-surgical muscle weakness) prolonged bed rest or stroke rehabilitation; 3) central nervous system (CNS) disorders in which muscle weakness, atrophy and fatigue are prominent symptoms, such as multiple sclerosis, Parkinson's disease, stroke and spinal cord injury; and 4) muscle symptoms stemming from systemic disorders, including Peripheral Vascular Disease (PVD) or Peripheral Arterial Disease (PAD) (e.g., claudication), metabolic syndrome, chronic fatigue syndrome, mobility limitation, obesity and frailty due to aging.

The polymorphic forms or compositions described herein may be used to treat neuromuscular diseases, i.e., diseases that affect any part of the nerve-muscle unit. Neuromuscular diseases include, for example: 1) diseases of the motor unit, including but not limited to Amyotrophic Lateral Sclerosis (ALS) including bulbar and primary lateral sclerosis (PLS) variants; spinal muscular atrophy types 1-4; Kennedy syndrome; post-polio syndrome; motor neuropathies including, for example, critical illness polyneuropathy; multifocal motor neuropathy with conduction block; Charcot-Marie-Tooth disease and other hereditary motor and sensory neuropathies; and Guillain-Barre Syndrome, 2) disorders of the neuromuscular junction, including myasthenia gravis, Lambert-Eaton myasthenic syndrome, and prolonged neuromuscular blockade due to drugs or toxins; and 3) peripheral neuropathies, such as acute inflammatory demyelinating polyradiculoneuropathy, diabetic neuropathy, chronic inflammatory demyelinating polyradiculoneuropathy, traumatic peripheral nerve lesions, neuropathy of leprosy, vasculitic neuropathy, dermatomyositis/polymyositis and neuropathy of Friedreich Ataxia.

The polymorphic forms or compositions described herein may be used to treat disorders of voluntary muscle. Disorders of voluntary muscle include 1) muscular dystrophies (including, for example, Duchenne, Becker, Limb-Girdle, Facioscapulohumeral, limb girdle, Emery-Dreyfus, oculopharyngeal and congenital muscular dystrophies); and 2) myopathies, such as nemaline myopathy, central core disease, congenital myopathies, mitochondrial myopathies, acute myopathy; inflammatory myopathies (such as dermatomyositis/polymyositis and inclusion body myositis), endocrine myopathies (such as those associated with hyper- or hypothyroidism), Cushing's or Addison's syndrome or disease and pituitary gland disorders, metabolic myopathies (such as glycogen storage diseases, e.g., McArdle's disease, Pompe disease, etc), drug-induced myopathy (statins, ant-retroviral drugs, steroid myopathy) restrictive lung disease, sarcoidosis, Schwartz-Jampel Syndrome, focal muscular atrophies, and distal myopathies.

The polymorphic forms or compositions described herein may be used to treat or Amyotrophic Lateral Sclerosis (ALS). ALS is a disease that generally arises later in life (Age 50+) and has a rapid progression from initial limb weakness to paralysis and death. Common life expectancy after diagnosis is 3-5 years. The cause of disease for most ALS patients is unknown (termed the spontaneous form) while a small proportion of patients have an inherited form (familial) of disease. The condition causes progressive death of motor neurons through causes that are not clear. Surviving motor units attempt to compensate for dying ones by innervating more fibers (termed sprouting) but this can only partially correct muscle function, as muscles are subsequently more prone to problems of coordination and fatigue. Eventually, surviving motor neurons die, resulting in complete paralysis of the affected muscle. The disease is commonly fatal through the eventual loss of innervation to the diaphragm, resulting in respiratory failure. Current treatment options for ALS are limited.

The polymorphic forms or compositions described herein may be used to treat Spinal Muscular Atrophy (SMA). SMA is a genetic disorder that arises through the mutation of a protein, SMN1, that appears to be required for the survival and health of motor neurons. The disease is most common in children as the majority of patients only survive until 11-12 years of age.

The polymorphic forms or compositions described herein may be used to treat myasthenia gravis. Myasthenia gravis is a chronic autoimmune neuromuscular disease wherein the body produces antibodies that block, alter, or destroy proteins involved in signaling at the neuromuscular junction, thus preventing muscle contraction from occurring. These proteins include nicotinic acetylcholine receptor (AChR) or, less frequently, a muscle-specific tyrosine kinase (MuSK) involved in AChR clustering (see, e.g., Drachman, N. Eng. J. of Med., 330:1797-1810, 1994). The disease is characterized by varying degrees of weakness of the skeletal (voluntary) muscles of the body. The hallmark of myasthenia gravis is muscle weakness that increases during periods of activity and improves after periods of rest. Although myasthenia gravis may affect any voluntary muscle, certain muscles, such as those that control eye and eyelid movement, facial expression, chewing, talking, and swallowing are often, but not always, involved in the disorder. The muscles that control breathing and neck and limb movements may also be affected. In most cases, the first noticeable symptom is weakness of the eye muscles. In others, difficulty in swallowing and slurred speech may be the first signs. The degree of muscle weakness involved in myasthenia gravis varies greatly among patients, ranging from a localized form, limited to eye muscles (ocular myasthenia), to a severe or generalized form in which many muscles—sometimes including those that control breathing—are affected. Symptoms, which vary in type and severity, may include a drooping of one or both eyelids (ptosis), blurred or double vision (diplopia) due to weakness of the muscles that control eye movements, unstable or waddling gait, weakness in arms, hands, fingers, legs, and neck, a change in facial expression, difficulty in swallowing and shortness of breath, and impaired speech (dysarthria). Generalized weakness develops in approximately 85% of patients.

The polymorphic forms or compositions described herein may be used to treat sarcopenia, e.g., sarcopenia associated with aging or disease (e.g. HIV infection). Sarcopenia is characterized by a loss of skeletal muscle mass, quality, and strength. Clinically, a decline in skeletal muscle tissue mass (muscle atrophy) contributes to frailty in older individuals. In human males, muscle mass declines by one-third between the ages of 50 and 80. In older adults, extended hospitalization can result in further disuse atrophy leading to a potential loss of the ability for independent living and to a cascade of physical decline. Moreover, the physical aging process profoundly affects body composition, including significant reductions in lean body mass and increases in central adiposity. The changes in overall adiposity and fat distribution appear to be important factors in many common age-related diseases such as hypertension, glucose intolerance and diabetes, dyslipidemia, and atherosclerotic cardiovascular disease. In addition, it is possible that the age-associated decrement in muscle mass, and subsequently in strength and endurance, may be a critical determinant for functional loss, dependence and disability. Muscle weakness is also a major factor predisposing the elderly to falls and the resulting morbidity and mortality.

The polymorphic forms or compositions described herein may be used to treat cachexia. Cachexia is a state often associated with cancer or other serious diseases or conditions, (e.g., chronic obstructive pulmonary disease, heart failure, chronic kidney disease, kidney dialysis), that is characterized by progressive weight loss, muscle atrophy and fatigue, due to the deletion of adipose tissue and skeletal muscle.

The polymorphic forms or compositions described herein may be used to treat muscular dystrophies. Muscular dystrophy can be characterized by progressive muscle weakness, destruction and regeneration of the muscle fibers, and eventual replacement of the muscle fibers by fibrous and fatty connective tissue.

The polymorphic forms or compositions described herein may be used to treat post-surgical muscle weakness, which is a reduction in the strength of one or more muscles following surgical procedure. Weakness may be generalized (i.e. total body weakness) or localized to a specific area, side of the body, limb, or muscle.

The polymorphic forms or compositions described herein may be used to treat post-traumatic muscle weakness, which is a reduction in the strength of one or more muscles following a traumatic episode (e.g. bodily injury). Weakness may be generalized (i.e. total body weakness) or localized to a specific area, side of the body, limb, or muscle.

The polymorphic forms or compositions described herein may be used to treat muscle weakness and fatigue produced by peripheral vascular disease (PVD) or peripheral artery disease (PAD). Peripheral vascular disease is a disease or disorder of the circulatory system outside of the brain and heart. Peripheral artery disease (PAD), also known as peripheral artery occlusive disease (PAOD), is a form of PVD in which there is partial or total blockage of an artery, usually one leading to a leg or arm. PVD and/or PAD can result from, for example, atherosclerosis, inflammatory processes leading to stenosis, embolus/thrombus formation, or damage to blood vessels due to disease (e.g., diabetes), infection or injury. PVD and/or PAD can cause either acute or chronic ischemia, typically of the legs. The symptoms of PVD and/or PAD include pain, weakness, numbness, or cramping in muscles due to decreased blood flow (claudication), muscle pain, ache, cramp, numbness or fatigue that occurs during exercise and is relieved by a short period of rest (intermittent claudication), pain while resting (rest pain) and biological tissue loss (gangrene). The symptoms of PVD and/or PAD often occur in calf muscles, but symptoms may also be observed in other muscles such as the thigh or hip. Risk factors for PVD and/or PAD include age, obesity, sedentary lifestyle, smoking, diabetes, high blood pressure, and high cholesterol (i.e., high LDL, and/or high triglycerides and/or low HDL). People who have coronary heart disease or a history of heart attack or stroke generally also have an increased frequency of having PVD and/or PAD. Activators of the fast skeletal troponin complex have been shown to reduce muscle fatigue and/or to increase the overall time to fatigue in in vitro and in situ models of vascular insufficiency (see, e.g., Russell et al., “The Fast Skeletal Troponin Activator, CK-2017357, Increases Skeletal Muscle Force and Reduces Muscle Fatigue in vitro and in situ”, 5th Cachexia Conference, Barcelona, Spain, December 2009; Hinken et al., “The Fast Skeletal Troponin Activator, CK-2017357, Reduces Muscle Fatigue in an in situ Model of Vascular Insufficiency”, Society for Vascular Medicine's 2010 Annual Meeting: 21st Annual Scientific Sessions, Cleveland, Ohio, April 2010).

The polymorphic forms or compositions described herein may be used to treat symptoms of frailty, e.g., frailty associated with aging. Frailty is characterized by one or more of unintentional weight loss, muscle weakness, slow walking speed, exhaustion, and low physical activity.

The polymorphic forms or compositions described herein may be used to treat muscle weakness and/or fatigue due to wasting syndrome, which is a condition characterized by involuntary weight loss associated with chronic fever and diarrhea. In some instances, patients with wasting syndrome lose 10% of baseline body weight within one month.

The polymorphic forms or compositions described herein may be used to treat muscular diseases and conditions caused by structural and/or functional abnormalities of skeletal muscle tissue, including muscular dystrophies, congenital muscular dystrophies, congenital myopathies, distal myopathies, other myopathies (e.g., myofibrillar, inclusion body), myotonic syndromes, ion channel muscle diseases, malignant hyperthermias, metabolic myopathies, congenital myasthenic syndromes, sarcopenia, muscle atrophy and cachexia.

The polymorphic forms or compositions described herein also may be used to treat diseases and conditions caused by muscle dysfunction originating from neuronal dysfunction or transmission, including amyotrophic lateral sclerosis, spinal muscular atrophies, hereditary ataxias, hereditary motor and sensory neuropathies, hereditary paraplegias, stroke, multiple sclerosis, brain injuries with motor deficits, spinal cord injuries, Alzheimer's disease, Parkinson's disease with motor deficits, myasthenia gravis and Lambert-Eaton syndrome.

The polymorphic forms or compositions described herein also may be used to treat diseases and conditions caused by CNS, spinal cord or muscle dysfunction originating from endocrine and/or metabolic dysregulation, including claudication secondary to peripheral artery disease, hypothyroidism, hyper- or hypo-parathyroidism, diabetes, adrenal dysfunction, pituitary dysfunction and acid/base imbalances.

The polymorphic forms or compositions described herein may be administered alone or in combination with other therapies and/or therapeutic agents useful in the treatment of the aforementioned disorders.

The polymorphic forms or compositions described herein may be combined with one or more other therapies to treat ALS. Examples of suitable therapies include riluzole, edaravone, baclofen, diazepam, trihexyphenidyl and amitriptyline. In some embodiments, the polymorphs and compositions described and/or disclosed herein are combined with riluzole to treat a subject suffering from ALS. In some embodiments, the polymorphs and compositions described and/or disclosed herein are combined with edaravone to treat a subject suffering from ALS.

The polymorphic forms or compositions described herein may be combined with one or more other therapies to treat SMA. Examples of suitable therapies include riluzole and nusinersen. In some embodiments, the polymorphs and compositions described and/or disclosed herein are combined with riluzole to treat a subject suffering from SMA. In some embodiments, the polymorphs and compositions described and/or disclosed herein are combined with nusinersen to treat a subject suffering from SMA.

The polymorphic forms or compositions described herein may be combined with one or more other therapies to treat myasthenia gravis. Examples of suitable therapies include administration of anticholinesterase agents (e.g., neostigmine, pyridostigmine), which help improve neuromuscular transmission and increase muscle strength; administration of immunosuppressive drugs (e.g., prednisone, cyclosporine, azathioprine, mycophenolate mofetil) which improve muscle strength by suppressing the production of abnormal antibodies; thymectomy (i.e., the surgical removal of the thymus gland, which often is abnormal in myasthenia gravis patients); plasmapheresis; and intravenous immune globulin.

The polymorphic forms or compositions described herein may be combined with one or more other therapies to treat PVD or PAD (e.g., claudication). Treatment of PVD and PAD is generally directed to increasing arterial blood flow, such as by smoking cessation, controlling blood pressure, controlling diabetes, and exercising. Treatment can also include medication, such as medicines to help improve walking distance (e.g., cilostazol, pentoxifylline), antiplatelet agents (e.g., aspirin, ticlopidine, clopidogrel), anticoagulents (e.g., heparin, low molecular weight heparin, warfarin, enoxaparin) throbmolytics, antihypertensive agents (e.g., diuretics, ACE inhibitors, calcium channel blockers, beta blockers, angiotensin II receptor antagonists), and cholesterol-lowering agents (e.g., statins). In some patients, angioplasty, stenting, or surgery (e.g., bypass surgery or surgery to remove an atherosclerotic plaque) may be necessary.

### Kits

Also provided are articles of manufacture and kits containing materials useful for the treatment of a disease or condition associated with neuromuscular or non-neuromuscular dysfunction, muscular weakness, and/or muscle fatigue. The article of manufacture may comprise a container with a label. Suitable containers include, for example, bottles, vials, and test tubes. The containers may be formed from a variety of materials such as glass or plastic. The container may hold a formulation having an active agent which is effective in treating a disease or condition associated with neuromuscular or non-neuromuscular dysfunction, muscular weakness, and/or muscle fatigue. The active agent in the formulation is one or more of the polymorphic forms described herein. The label on the container may indicate that the formulation is used for treating a disease or condition associated with neuromuscular or non-neuromuscular dysfunction, muscular weakness, and/or muscle fatigue, and may also indicate directions for either in vivo or in vitro use, such as those described above.

Also provided are kits containing any one or more of the polymorphic forms or compositions described herein. In some embodiments, the kit comprises the container described above. In other embodiments, the kit comprises the container described above and a second container comprising a buffer. It may further include other materials desirable from a commercial and user standpoint, including other buffers, diluents, filters, needles, syringes, and package inserts with instructions for performing any methods described herein.

In other aspects, the kits may be used for any of the methods described herein, including, for example, to treat an individual with a disease or condition associated with neuromuscular or non-neuromuscular dysfunction, muscular weakness, and/or muscle fatigue.

In certain embodiments the kits may include a dosage amount of at least one formulation as disclosed herein. Kits may also comprise a means for the delivery of the formulation thereof.

The kits may include other pharmaceutical agents for use in conjunction with the formulation described herein. In some variations, the pharmaceutical agent(s) may be one or more anti-psychotic drugs. These agents may be provided in a separate form, or mixed with the compounds described herein, provided such mixing does not reduce the effectiveness of either the pharmaceutical agent or formulation described herein and is compatible with the route of administration. Similarly, the kits may include additional agents for adjunctive therapy or other agents known to the skilled artisan as effective in the treatment or prevention of the conditions described herein.

The kits may optionally include appropriate instructions for preparation and administration of the formulation, side effects of the formulation, and any other relevant information. The instructions may be in any suitable format, including, but not limited to, printed matter, videotape, computer readable disk, optical disc or directions to internet-based instructions.

In another aspect, kits for treating an individual who suffers from or is susceptible to the conditions described herein are provided, comprising a first container comprising a dosage amount of a composition as disclosed herein, and instructions for use. The container may be any of those known in the art and appropriate for storage and delivery of intravenous formulation. In certain embodiments, the kit further comprises a second container comprising a pharmaceutically acceptable carrier, diluent, adjuvant, etc. for preparation of the formulation to be administered to the individual.

Kits may also be provided that contain sufficient dosages of the polymorphs described herein (including formulations thereof) to provide effective treatment for an individual for an extended period, such as 1-3 days, 1-5 days, a week, 2 weeks, 3, weeks, 4 weeks, 6 weeks, 8 weeks, 3 months, 4 months, 5 months, 6 months, 7 months, 8 months, 9 months or more.

Kits may also include multiple doses of the formulation and instructions for use and may be packaged in quantities sufficient for storage and use in pharmacies, for example, hospital pharmacies and compounding pharmacies.

The kits may include the composition as described herein packaged in either a unit dosage form or in a multi-use form. The kits may also include multiple units of the unit dose form.

In certain embodiments are provided a formulation described herein in a unit dose form. In other embodiments a formulation may be provided in a multi-dose form (e.g., a blister pack, etc.).

### EXAMPLES

The following examples are provided to further aid in understanding the embodiments disclosed in the application, and presuppose an understanding of conventional methods well known to those persons having ordinary skill in the art to which the examples pertain. The particular materials and conditions described hereunder are intended to exemplify particular aspects of embodiments disclosed herein and should not be construed to limit the reasonable scope thereof.

The polymorphic forms of 1-(2-((((trans)-3-fluoro-1-(3-fluoropyridin-2-yl)cyclobutyl)methyl)amino)pyrimidin-5-yl)-1H-pyrrole-3-carboxamide were characterized by various analytical techniques, including X-ray powder diffraction (XPPD), differential scanning calorimetry (DSC), and thermographic analysis (TGA) using the procedures described below.

Ray Powder Diffraction patterns were collected on a Bruker D8 diffractometer using Cu Kα radiation (40 kV, 40 mA), θ-2θ goniometer, and divergence of V4 and receiving slits, a Ge monochromator and a Lynxeye detector. The instrument is performance checked using a certified Corundum standard (NIST 1976). The software used for data collection was Diffrac Plus XRD Commander v2.6.1 and the data were analysed and presented using Diffrac Plus EVA v13.0.0.2 or v15.0.0.0.

Samples were run under ambient conditions as flat plate specimens using powder as received. The sample was gently packed into a cavity cut into polished, zero-background (510) silicon wafer. The sample was rotated in its own plane during analysis. The details of the data collection are:
Angular range: 2 to 42° 2θ
Step size: 0.05° 2θ
Collection time: 0.5 s/step

DSC data were collected on a TA Instruments Q2000 equipped with a 50 position auto-sampler. The calibration for thermal capacity was carried out using sapphire and the calibration for energy and temperature was carried out using certified indium. Typically 0.5-3 mg of each sample, in a pin-holed aluminum pan, was heated at 10° C./min from 25° C. to 300° C. A purge of dry nitrogen at 50 ml/min was maintained over the sample. Modulated temperature DSC was carried out using an underlying heating rate of 2° C./min and temperature modulation parameters of ±0.636° C. (amplitude) every 60 seconds (period). The instrument control software was Advantage for Q Series v2.8.0.394 and Thermal Advantage v5.2.6 and the data were analysed using Universal Analysis v4.7A or v4.4A.

TGA data were collected on a Mettler TGA/SDTA 851e equipped with a 34 position auto-sampler. The instrument was temperature calibrated using certified indium. Typically 5-30 mg of each sample was loaded onto a pre-weighed aluminum crucible and was heated at 10° C./min from ambient temperature to 350° C. A nitrogen purge at 50 ml/min was maintained over the sample. The instrument control and data analysis software was STARe v9.20.

GVS data were collected using a SMS DVS Intrinsic moisture sorption analyser, controlled by DVS Intrinsic Control software v1.0.0.30. The sample temperature was maintained at 25° C. by the instrument controls. The humidity was controlled by mixing streams of dry and wet nitrogen, with a total flow rate of 200 ml/min The relative humidity was measured by a calibrated Rotronic probe (dynamic range of 1.0-100% RH), located near the sample. The weight change, (mass relaxation) of the sample as a function of % RH was constantly monitored by the microbalance (accuracy ±0.005 mg).

Typically 5-20 mg of sample was placed in a tared mesh stainless steel basket under ambient conditions. The sample was loaded and unloaded at 40% RH and 25° C. (typical room conditions). A moisture sorption isotherm was performed as outlined below (2 scans giving 1 complete cycle). The standard isotherm was performed at 25° C. at 10% RH intervals over a 0-90% RH range. Data analysis was undertaken in Microsoft Excel using DVS Analysis Suite v6.0.

Method Parameters for SMS DVS Intrinsic Experiments

| Parameters | Values |
|---|---|
| Adsorption - Scan 1 | 40-90 |
| Desorption/Adsorption - Scan 2 | 90-0, 0-40 |
| Intervals (% RH) | 10 |
| Number of Scans | 4 |
| Flow rate (ml/min) | 200 |
| Temperature (° C.) | 25 |
| Stability (° C./min) | 0.2 |
| Sorption Time (hours) | 6 hour time out |

### Example 1. Polymorph Screening

Polymorphic Form I of 1-(2-((((trans)-3-fluoro-1-(3-fluoropyridin-2-yl)cyclobutyl)methyl)amino)pyrimidin-5-yl)-1H-pyrrole-3-carboxamide was weighed into vials, and the stated solvents were added, in increasing portions, at 50° C. With each addition, the vials were allowed to shake for ˜20 minutes. After the last addition, any suspensions observed were subjected to heat/cool cycles, between room temperature and 50° C., 4 hours at each condition, for 1 week. Solutions were allowed to slowly evaporate.

Most experiments showed suspensions in up to 50 volumes solvent. Aliquots were filtered by suction after 7 days maturation and analysed by XRPD (low resolution, air-dried only). Most solutions yielded solids after 9 days and these were analyzed by XRPD as well. The results of the polymorph screening are shown in Table 6.

TABLE 6
Polymorph screening in 24 solvents

| | Solubility (volumes) | | | | |
|---|---|---|---|---|---|
| | (✓ YES; x NO) | | | | |
| | at 50° C. | | | | |
| Solvent | 10 | 25 | 50 | Procedure | XRPD |
| Toluene | x | x | x | Temperature | Form I |
| | | | | cycle | |
| Anisole | x | x | x | Temperature | DIFFERENT |
| | | | | cycle | |
| Heptane | x | x | x | Temperature | DIFFERENT |
| | | | | cycle | |
| 1,4-Dioxane | x | x | x | Temperature | Form III |
| | | | | cycle | |
| Tert-butyl methyl | x | x | x | Temperature | DIFFERENT |
| ether (TBME) | | | | cycle | |
| Butyl acetate | x | x | x | Temperature | Form I |
| (BuOAc) | | | | cycle | |
| Isopropyl acetate | x | x | x | Temperature | Form I |
| (IPAc) | | | | cycle | |
| Ethyl acetate | x | x | x | Temperature | DIFFERENT |
| | | | | cycle | |
| Methyl isobutyl | x | x | x | Temperature | DIFFERENT |
| ketone (MIBK) | | | | cycle | |
| Methyl ethyl | x | x | x | Temperature | Form I |
| ketone (MEK) | | | | cycle | |
| Acetone | x | x | x | Temperature | DIFFERENT |
| | | | | cycle | |
| Ethanol | x | x | x | Temperature | DIFFERENT |
| | | | | cycle | |
| Methanol | x | x | x | Temperature | Form I |
| | | | | cycle | |
| 1-Butanol | x | x | x | Temperature | Form I |
| | | | | cycle | |
| 1-Propanol | x | x | x | Temperature | Form I |
| | | | | cycle | |
| 2-Propanol | x | x | x | Temperature | Form I |
| | | | | cycle | |
| Acetonitrile | ✓ | n/a | n/a | Evaporation | n/a |
| Tetrahydrofuran | x | x | x | Temperature | DIFFERENT |
| (THF) | | | | cycle | |
| Nitromethane | x | x | x | Temperature | Amorphous |
| | | | | cycle | |
| Water | x | x | x | Temperature | Form I |
| | | | | cycle | |
| Dioxane/ | x | x | ✓ | Evaporation | Form III |
| 5% water | | | | | |
| Ethanol/ | x | x | x | Temperature | Form I |
| 5% water | | | | cycle | |
| Isopropanol | x | x | x | Temperature | Form I |
| (IPA) | | | | cycle | |
| /5% water | | | | | |
| THF/ | x | Cloudy | ✓ | Evaporation | Form II |
| 5% water | | | | | |
| Dichloromethane | x | x | x | Temperature | Form I |
| (DCM) | | | | cycle | |

“DIFFERENT” means that the XRPD pattern is different from that of Form I, II, or III.

Those experiments producing XRPD patterns other than those consistent with Form I were repeated on a larger scale. Form I (˜40 mg) was weighed into vials, and the stated solvents were added (50 volumes). Any suspensions observed were subjected to heat/cool cycles, between room temperature and 50° C., four hours at each condition. Solutions were allowed to slowly evaporate. All solids obtained were analyzed by XRPD after 5 and 20 days maturation. The results are shown in Table 7.

TABLE 7

| Solvent | | XRPD | XRPD |
|---|---|---|---|
| (50 volumes) | Procedure | 5 days | 20 days |
| Anisole | Temperature | Form I | Form I |
| | cycle | | |
| Heptane | Temperature | Form I | Form I |
| | cycle | | |
| 1,4-Dioxane | Temperature | Form III | n/a |
| | cycle | | |
| TBME | Temperature | Form I | Form I |
| | cycle | | |
| MIBK | Temperature | Form I | Form I |
| | cycle | | |
| MEK | Temperature | Form I | Dissolved |
| | cycle | | after 20 days |
| | | | n/a |
| Acetone | Temperature | Slightly | n/a |
| | cycle | different | |
| | | pattern | |
| Ethanol | Temperature | Form I | Form I |
| | cycle | | |
| Acetonitrile | Temperature | Form I | Form I |
| | cycle | | |
| THF | Temperature | Form I | Pattern 3 |
| | cycle | | |
| Nitromethane | Temperature | Form I | Form IV |
| | cycle | | |
| Dioxane/ | Dissolved at | n/a | Form I |
| 10% water | 50° C. - | | |
| | evaporation | | |
| THF/ | Temperature | Form I | Pattern 3 |
| 10% water | cycle | | |
| Methanol | Dissolved after | n/a | Form I |
| | 4 days cycling - | | |
| | Evaporation | | |

Polymorphic Form I of 1-(2-((((trans)-3-fluoro-1-(3-fluoropyridin-2-yl)cyclobutyl)methyl)amino)pyrimidin-5-yl)-1H-pyrrole-3-carboxamide was analyzed by XRPD, DSC, TGA, and GVS. FIG. 1A shows experimental and simulated XRPD patterns of Form I. An XRPD pattern that may be observed for Form I is also shown in FIG. 12. FIG. 1B shows DSC and TGA graphs of Form I. FIG. 1C shows a GVS graph of Form I. As shown in FIG. 1C, Form I is not hygroscopic, showing less than 0.07% moisture uptake over 0-90% relative humidity (RH) range as determined by GVS. Chemical stability study of Form I was also performed. XRPD patterns were measured after storage of samples of Form I for 7 days at 40° C./75% RH and 25° C./97% RH. No visible changes in XRPD patterns after storage were observed.

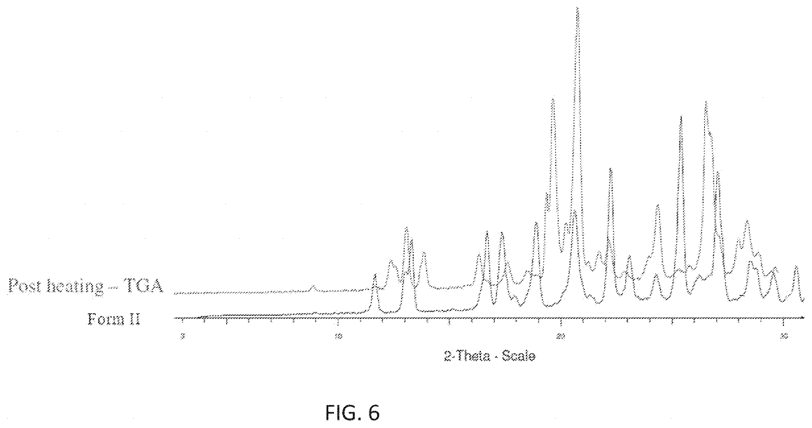

Polymorphic Form II of 1-(2-((((trans)-3-fluoro-1-(3-fluoropyridin-2-yl)cyclobutyl)methyl)amino)pyrimidin-5-yl)-1H-pyrrole-3-carboxamide was analyzed by XRPD, DSC, TGA, and GVS. FIG. 2A shows experimental and simulated XRPD patterns of Form II. An XRPD pattern that may be observed for Form II is also shown in FIG. 12. FIG. 2B shows DSC and TGA graphs of Form II. FIG. 2C shows a GVS graph of Form II. As shown in FIG. 2C, Form II was observed to take up about 1.65% water over 0-90% RH as determined by GVS. XRPD analysis of Form II after heating at 150° C. indicated that Form II was converted to Form I, as shown in FIG. 6.

Polymorphic Form III of 1-(2-((((trans)-3-fluoro-1-(3-fluoropyridin-2-yl)cyclobutyl)methyl)amino)pyrimidin-5-yl)-1H-pyrrole-3-carboxamide dioxane solvate was analyzed by XRPD, DSC, and TGA. FIG. 3A shows experimental and simulated XRPD patterns of Form III. An XRPD pattern that may be observed for Form III is also shown in FIG. 12. FIG. 3B shows DSC and TGA graphs of Form III.

Polymorphic Form IV of 1-(2-((((trans)-3-fluoro-1-(3-fluoropyridin-2-yl)cyclobutyl)methyl)amino)pyrimidin-5-yl)-1H-pyrrole-3-carboxamide was re-prepared by desolvation of a THF solvate in order to perform further characterization. Form I (˜250 mg) was suspended in THF (20 volumes) at 40° C., and allowed to shake at this temperature for 5 days. An aliquot was then filtered and XRPD was performed, confirming the formation of the THF solvate, Pattern 3. The remaining solid was isolated by suction. Small portions of the THF solvate were placed in a TGA pan and heated to 120° C. and held isothermally for 5 minutes in the TGA instrument. Analysis by XRPD post heating confirmed the formation of Form IV. Form IV was analyzed by XRPD. FIG. 4 shows XRPD patterns of Form IV prepared using different methods (top: from desolvation of Form III in TGA; middle: from maturation of Form I in nitromethane; bottom: from desolvation of Form III in variable temperature-XRPD). An XRPD pattern that may be observed for Form IV is also shown in FIG. 12.

### Example 2. Preparation of Form V

Mixtures of Form I and Form II were reproducibly converted to pure Form V by slurrying at 45-50° C. in ethanol, denatured ethanol or aqueous denatured ethanol. The time needed to achieve the conversion depended on the amount of Form V initially present in the sample (conversion of a mixture Form I/Form II containing traces of Form V was achieved in 4 to 12 hours).

The following 2-step crystallisation protocol was used to prepare pure Form V exhibiting a high chemical purity (99%, 0.1% w/w residual ethanol) with a yield of 85% on a 7.5 g scale:

Step 1:
addition of 25 volumes of 25:75% vol. water:denatured ethanol to 1-(2-((((trans)-3-fluoro-1-(3-fluoropyridin-2-yl)cyclobutyl)methyl)amino)pyrimidin-5-yl)-1H-pyrrole-3-carboxamide, stirring at 400 RPM;
temperature increased to 70° C. (0.5° C./min), 10 minutes at 70° C. (complete dissolution);
cooling to 60° C. at 0.2° C./min;
seeding with 1.0% w/w Form V, 30 minutes at 60° C.;
cooling to 45° C. at 0.2° C./min;
slurrying at 45° C. for 5 hours
cooling to 0° C. at 0.2° C./min;
filtration and drying: Form V+Form I and Form II, 90% yield.

Step 2:
addition of 4 volumes of denatured ethanol to the material obtained after Step 1, stirring at 300 RPM;
temperature increased to 50° C. (0.5° C./min);
slurrying at 50° C.; sampling for control by XRPD: pure FormV after 3 hours filtration and drying: Form V, 85% yield.

An alternative process of preparing Form V is provided below.

1-(2-((((trans)-3-fluoro-1-(3-fluoropyridin-2-yl)cyclobutyl)methyl)amino)pyrimidin-5-yl)-1H-pyrrole-3-carboxamide was mixed with 75% (v/v) 1-propanol/water (10V) and agitated. The mixture was heated to 85° C. and agitated until a complete solution was obtained. The solution was cooled to 70° C. at a rate no more than 10° C./h. At 72° C., seed crystal (0.1 wt %) was added as a suspension in 1-propanol. The resulting suspension was agitated at 70° C. until the concentration of 1-(2-((((trans)-3-fluoro-1-(3-fluoropyridin-2-yl)cyclobutyl)methyl)amino)pyrimidin-5-yl)-1H-pyrrole-3-carboxamide in supernatant determined by HPLC was <85 mg/mL. The resulting suspension was then cooled to 40° C. at a rate no more than 5° C./h and agitated until the concentration of 1-(2-((((trans)-3-fluoro-1-(3-fluoropyridin-2-yl)cyclobutyl)methyl)amino)pyrimidin-5-yl)-1H-pyrrole-3-carboxamide in supernatant was <24 mg/mL. The resulting suspension was then cooled to 20° C. at a rate no more than 5° C./h and agitated until the concentration of 1-(2-((((trans)-3-fluoro-1-(3-fluoropyridin-2-yl)cyclobutyl)methyl)amino)pyrimidin-5-yl)-1H-pyrrole-3-carboxamide in supernatant was <12 mg/mL. The resulting suspension was cooled to 0° C. at a rate no more than 5° C./h and agitated until the concentration of 1-(2-((((trans)-3-fluoro-1-(3-fluoropyridin-2-yl)cyclobutyl)methyl)amino)pyrimidin-5-yl)-1H-pyrrole-3-carboxamide in the supernatant was <7 mg/mL. The resulting crystalline solids were filtered and dried (93% yield).

Polymorphic Form V of 1-(2-((((trans)-3-fluoro-1-(3-fluoropyridin-2-yl)cyclobutyl)methyl)amino)pyrimidin-5-yl)-1H-pyrrole-3-carboxamide was analyzed by XRPD, DSC, TGA, and GVS. FIG. 5A shows an experimental XRPD pattern of Form V. An XRPD pattern that may be observed for Form V is also shown in FIG. 12. FIG. 5B shows DSC and TGA graphs of Form V. FIG. 5C shows a GVS graph of Form V. As shown in FIG. 5C, Form V is not hygroscopic, showing less than 0.05% moisture uptake over 0-90% RH range as determined by GVS.

### Example 3. Competitive Slurry Experiments Between Form I and Form II

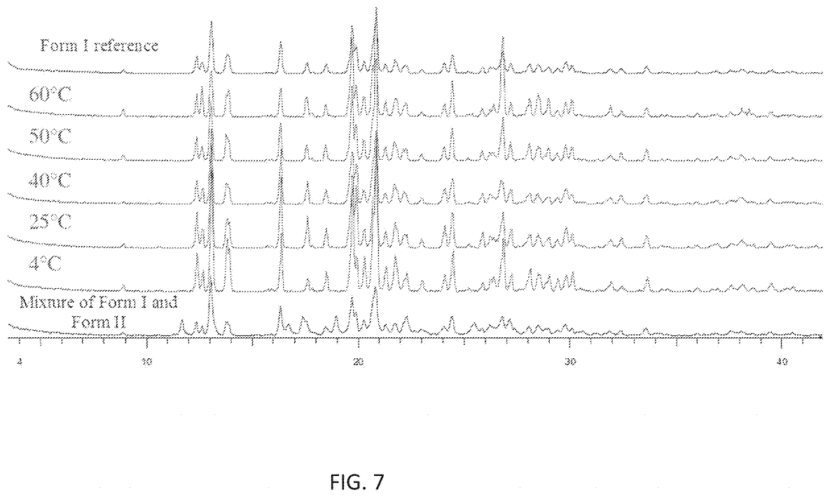

A mechanical mixture of Form I and Form II was prepared. This mixture was analyzed by XRPD for reference. The mixture (ca. 25 mg) was suspended in methanol, and parallel maturation experiments were set up at a constant temperature, in the range of 4 to 60° C., for a total of 12 days. The resulting solids were filtered and analyzed by XRPD. The results are shown in FIG. 7. The five experiments showed pure Form I after 12 days of maturation, indicating that Form I is more stable in this temperature range.

### Example 4. Competitive Slurry Experiments Between Form I and Form IV

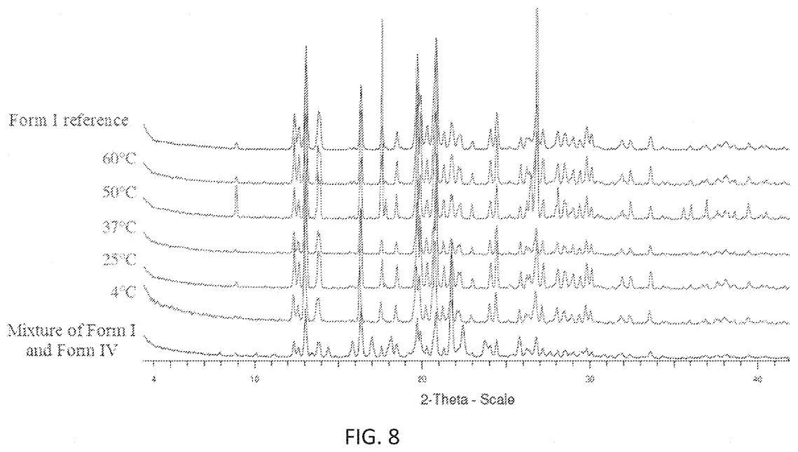

Competitive slurry experiments between Form I and Form IV were conducted in a manner similar to that described in Example 3. The results are shown in FIG. 8. The five experiments showed pure Form I after 12 days of maturation, indicating that Form I is more stable in this temperature range.

### Example 5. Competitive Slurry Experiments Between Form I and Form V

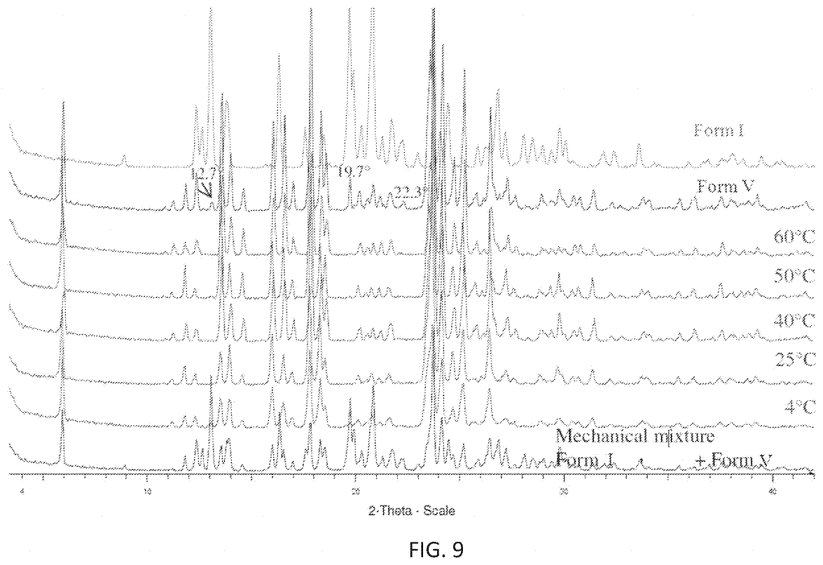

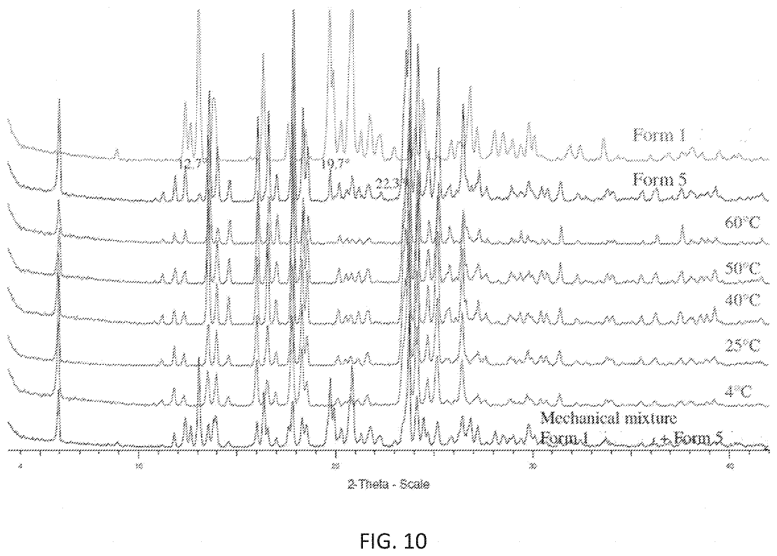

For competitive slurry experiments, a mixture of Form I (˜10 mg) and Form V (˜10 mg) were suspended in 20 volumes of ethanol or methanol. Parallel maturation experiments were set up at a constant temperature, in the range of 4° C. to 60° C. for 9-12 days, the resulting solids (rapidly isolated by solvent decantation) were analyzed by XRPD. A mechanical mixture of Form I and Form V in equivalent weight was also analyzed by XRPD for reference. For the control experiments, slurries of Form I or Form V were subjected to the same conditions as in the competitive slurry experiments. The results of the competitive slurry experiments in ethanol are shown in FIG. 9. XRPD results showed only Form V after 9-12 days of maturation, indicating that Form V is more stable in this temperature range. The results of the competitive slurry experiments in methanol are shown in FIG. 10. The results also indicated that Form V is more stable in this temperature range.

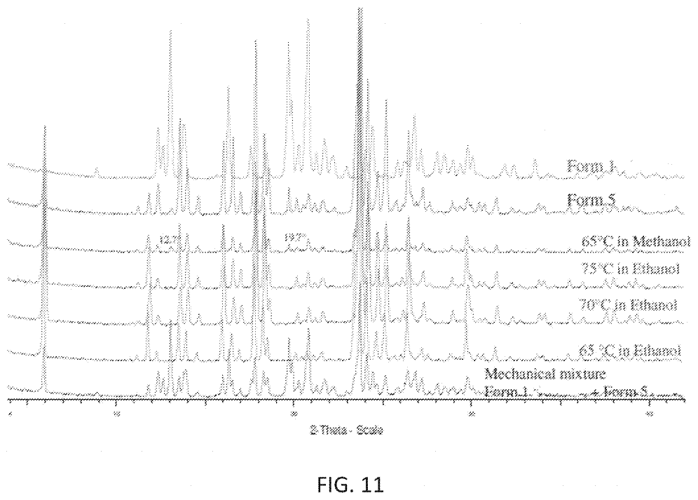

In an attempt to determine a transition temperature for the polymorph pair, a further four competitive slurry experiments were performed at a higher temperature range of 65-75° C. Using sealed vials, Form I (˜10 mg) and Form V (˜10 mg) were suspended in 20 volumes of ethanol or methanol and maturation continued for 2 days. The results are shown in FIG. 11. All solid produced using ethanol were consistent with Form V, indicating Form V is the more stable form in ethanol in this temperature range. The sample in methanol at 65° C. gave a mixture of both forms.

All documents, including patents, patent application and publications cited herein, including all documents cited therein, tables, and drawings, are hereby expressly incorporated by reference in their entirety for all purposes.

While the foregoing written description of the compounds, uses, and methods described herein enables one of ordinary skill in the art to make and use the compounds, uses, and methods described herein, those of ordinary skill in the art will understand and appreciate the existence of variations, combinations, and equivalents of the specific embodiment, method, and examples herein. The compounds, uses, and methods provided herein should therefore not be limited by the above-described embodiments, methods, or examples, but rather encompasses all embodiments and methods within the scope and spirit of the compounds, uses, and methods provided herein.

1. A polymorph of 1-(2-((((trans)-3-fluoro-1-(3-fluoropyridin-2-yl)cyclobutyl)methyl)amino)pyrimidin-5-yl)-1H-pyrrole-3-carboxamide.

2. The polymorph of claim 1, characterized by having an XRPD pattern comprising peaks at angles 2-theta of 13.0±0.2, 16.3±0.2, 19.7±0.2, 19.9±0.2, and 20.8±0.2 degrees.

3. The polymorph of claim 1 or 2, characterized by having an XRPD pattern comprising peaks at angles 2-theta of 12.3±0.2, 13.0±0.2, 13.8±0.2, 16.3±0.2, 19.7±0.2, 19.9±0.2, 20.8±0.2, 21.7±0.2, 24.5±0.2, and 26.8±0.2 degrees.

4. The polymorph of any one of claims 1-3, characterized by having an XRPD pattern substantially as shown in FIG. 1A or FIG. 12.

5. The polymorph of any one of claims 1-4, characterized by having a DSC graph substantially as shown in FIG. 1B.

6. The polymorph of any one of claims 1-5, characterized by having a melting endotherm onset at about 192° C. as determined by DSC.

7. The polymorph of any one of claims 1-6, characterized by having a TGA graph substantially as shown in FIG. 1B.

8. The polymorph of any one of claims 1-7, characterized by having a GVS graph substantially as shown in FIG. 1C.

9. The polymorph of claim 1, characterized by having an XRPD pattern comprising peaks at angles 2-theta of 11.6±0.2, 13.0±0.2, 17.4±0.2, 18.9±0.2, and 22.3±0.2 degrees.

10. The polymorph of claim 1 or 9, characterized by having an XRPD pattern comprising peaks at angles 2-theta of 11.6±0.2, 13.0±0.2, 13.2±0.2, 16.6±0.2, 17.4±0.2, 18.9±0.2, 20.7±0.2, 22.3±0.2, 25.5±0.2, and 27.1±0.2 degrees.

11. The polymorph of any one of claims 1, 9, and 10, characterized by having an XRPD pattern substantially as shown in FIG. 2A.

12. The polymorph of any one of claims 1 and 9-11, characterized by having a DSC graph substantially as shown in FIG. 2B.

13. The polymorph of any one of claims 1 and 9-12, characterized by having a melting endotherm onset at about 191° C. as determined by DSC.

14. The polymorph of any one of claims 1 and 9-13, characterized by having a TGA graph substantially as shown in FIG. 2B.

15. The polymorph of any one of claims 1 and 9-14, characterized by having a GVS graph substantially as shown in FIG. 2C.

16. The polymorph of claim 1, characterized by having an XRPD pattern comprising peaks at angles 2-theta of 7.6±0.2, 15.1±0.2, 18.1±0.2, 21.3±0.2, and 26.8±0.2 degrees.

17. The polymorph of claim 1 or 16, characterized by having an XRPD pattern comprising peaks at angles 2-theta of 7.6±0.2, 15.1±0.2, 18.1±0.2, 18.6±0.2, 19.4±0.2, 20.0±0.2, 21.3±0.2, 23.8±0.2, 25.1±0.2, and 26.8±0.2 degrees.

18. The polymorph of any one of claims 1, 16, and 17, characterized by having an XRPD pattern substantially as shown in FIG. 3A.

19. The polymorph of any one of claims 1 and 16-18, characterized by having a DSC graph substantially as shown in FIG. 3B.

20. The polymorph of any one of claims 1 and 16-19, characterized by having a broad endotherm with onset at about 75° C. and/or a melting endotherm onset at about 193° C. as determined by DSC.

21. The polymorph of any one of claims 1 and 16-20, characterized by having a TGA graph substantially as shown in FIG. 3B.

22. The polymorph of any one of claims 1 and 16-21, characterized by having a weight loss of about 23.8% w/w below 120° C. as determined by TGA.

23. The polymorph of claim 1, characterized by having an XRPD pattern comprising peaks at angles 2-theta of 16.4±0.2, 17.0±0.2, 18.1±0.2, 21.8±0.2, and 22.4±0.2 degrees.

24. The polymorph of claim 1 or 23, characterized by having an XRPD pattern comprising peaks at angles 2-theta of 14.4±0.2, 16.4±0.2, 17.0±0.2, 18.1±0.2, 18.6±0.2, 21.8±0.2, 22.4±0.2, 23.8±0.2, 25.8±0.2, and 31.7±0.2 degrees.

25. The polymorph of any one of claims 1, 23, and 24, characterized by having an XRPD pattern substantially as shown in FIG. 4.

26. The polymorph of claim 1, characterized by having an XRPD pattern comprising peaks at angles 2-theta of 17.8±0.2, 23.6±0.2, 23.7±0.2, 24.2±0.2, and 25.2±0.2 degrees.

27. The polymorph of claim 1 or 26, characterized by having an XRPD pattern comprising peaks at angles 2-theta of 5.9±0.2, 13.6±0.2, 16.6±0.2, 17.8±0.2, 18.4±0.2, 23.6±0.2, 23.7±0.2, 24.2±0.2, 25.2±0.2, and 26.5±0.2 degrees.

28. The polymorph of any one of claims 1, 26, and 27, characterized by having an XRPD pattern substantially as shown in FIG. 5A.

29. The polymorph of any one of claims 1 and 26-28, characterized by having a DSC graph substantially as shown in FIG. 5B.

30. The polymorph of any one of claims 1 and 26-29, characterized by having a melting endotherm onset at about 190° C. as determined by DSC.

31. The polymorph of any one of claims 1 and 26-30, characterized by having a TGA graph substantially as shown in FIG. 5B.

32. The polymorph of any one of claims 1 and 26-31, characterized by having a GVS graph substantially as shown in FIG. 5C.

33. A composition comprising a polymorph of any one of claims 1-32 and a pharmaceutically acceptable carrier.

34. A method of preparing the polymorph of any one of claims 2-8, comprising:
(a) mixing 1-(2-((((trans)-3-fluoro-1-(3-fluoropyridin-2-yl)cyclobutyl)methyl)amino)pyrimidin-5-yl)-1H-pyrrole-3-carboxamide with a solvent, wherein the solvent is selected from the group consisting of toluene, anisole, heptane, tert-butyl methyl ether (TBME), methyl isobutyl ketone (MIBK), methyl ethyl ketone (MEK), ethanol, acetonitrile, methanol, butyl acetate (BuOAc), isopropyl acetate (IPAc), 1-butanol, 1-propanol, 2-propanol, methylene dichloride (DCM), water, ethanol/5% water, and isopropyl alcohol (IPA)/5% water; and
(b) subjecting the mixture generated in step (a) to heat/cool cycles.

35. The method of claim 34, wherein the heat/cool cycles comprises cycles between room temperature and about 50° C., wherein the duration of each condition is about four hours.

36. A method of preparing the polymorph of any one of claims 9-15, comprising:
(a) mixing polymorphic Form I of 1-(2-((((trans)-3-fluoro-1-(3-fluoropyridin-2-yl)cyclobutyl)methyl)amino)pyrimidin-5-yl)-1H-pyrrole-3-carboxamide with a solvent, wherein the solvent is THF/5% water (v/v); and
(b) evaporating the mixture of step (a).

37. A method of preparing the polymorph of any one of claims 16-22, comprising:
(a) mixing Form I of 1-(2-((((trans)-3-fluoro-1-(3-fluoropyridin-2-yl)cyclobutyl)methyl)amino)pyrimidin-5-yl)-1H-pyrrole-3-carboxamide with a solvent, wherein the solvent is dioxane/5% water at a temperature of about 50° C.; and
(b) evaporating the mixture of step (a).

38. A method of preparing the polymorph of any one of claims 23-25, comprising:
(a) mixing polymorphic Form I of 1-(2-((((trans)-3-fluoro-1-(3-fluoropyridin-2-yl)cyclobutyl)methyl)amino)pyrimidin-5-yl)-1H-pyrrole-3-carboxamide with THF at a temperature of about 40° C., thereby generating a solid; and
(b) heating the solid generated in step (a).

39. A method of preparing the polymorph of any one of claims 26-32, comprising:
(a) mixing polymorphic Form I of 1-(2-((((trans)-3-fluoro-1-(3-fluoropyridin-2-yl)cyclobutyl)methyl)amino)pyrimidin-5-yl)-1H-pyrrole-3-carboxamide with aqueous 1-propanol, ethanol, denatured ethanol or aqueous denatured ethanol; and
(b) slurring the mixture of step (a).

40. A method of treating a disease associated with neuromuscular or non-neuromuscular dysfunction, muscular weakness, and/or muscle fatigue in a subject in need thereof, comprising administering to the subject a therapeutically effective amount of the polymorph of any one of claims 1-32 or the composition of claim 33.

41. The method of claim 40, wherein the disease is Amyotrophic Lateral Sclerosis (ALS), Spinal Muscular Atrophy (SMA), mobility limitation, Chronic Obstructive Pulmonary Disease (COPD), or myasthenia gravis.

42. A kit comprising a therapeutically effective amount of the polymorph of any one of claims 1-32 or the composition of claim 33.
